(19) United States
(12) Patent Application Publication
(10) Pub. No.: US 2026/0315129 A1
(43) Pub. Date: Oct. 15, 2026

(54) METHOD AND DEVICE FOR INTRAVASCULAR ABLATION FOR TREATMENT OF VASCULATURE

(71) Applicant: University of Massachusetts, Westborough, MA (US)

(72) Inventors: Govindarajan Srimathveeravalli, Westborough, MA (US); Poompavai Sadasivam, Westborough, MA (US); Brian Simoes, Westborough, MA (US); Neeraj Raghuraman Rajagopalan, Westborough, MA (US)

(21) Appl. No.: 19/636,623

(22) Filed: Apr. 1, 2026

Related U.S. Application Data
(60) Provisional application No. 63/781,774, filed on Apr. 1, 2025.
(60) Provisional application No. 63/892,470, filed on Oct. 2, 2025.

(51) Int. Cl. A61B 18/00; A61B 90/00

(52) U.S. Cl. CPC A61B 18/00; A61B 90/37; A61B 2018/0016; A61B 2018/00184; A61B 2018/00267; A61B 2018/00404; A61B 2018/00577; A61B 2018/00642; A61B 2018/00738; A61B 2018/00744; A61B 2018/00839; A61B 2018/0091

(57) ABSTRACT

A system and method for intravascular pulsed electric field treatment in a blood vessel, comprising an intravascular catheter comprising an expandable electrode assembly having a plurality of electrodes arranged in a basket configuration, each of the electrodes being (i) stowable within the catheter in a first configuration and (ii) expandable to a second configuration to conform to an inner wall of a blood vessel of varying diameter without occluding the blood vessel; a fluid delivery lumen configured to be positionable into the blood vessel concurrent with the intravascular catheter without occluding the blood vessel to introduce a conductive fluid into the blood vessel; and a controller coupled to (i) the fluid delivery lumen and the (ii) the plurality of electrodes, wherein the controller is configured to direct conductive fluid to the at least one fluid delivery lumen to displace blood at a treatment site and establish a localized conductive environment.

### RELATED APPLICATION

This application claims priority to, and the benefit of, U.S. Provisional Patent Application No. 63/781,774, filed Apr. 1, 2025, and U.S. Provisional Patent Application No. 63/892,470, filed Oct. 2, 2025, which are incorporated by reference herein in their entirety.

### GOVERNMENT SPONSORSHIP CLAUSE

This invention was made with government support under Grant No. 2338949 awarded by the National Science Foundation. The government has certain rights in the invention.

### BACKGROUND

Pulsed electrical field (PEF) ablation is a non-thermal-based treatment to perform cardiac ablation by delivering a series of punctuated electrical energy applications to tissue to modulate the cellular and interstitial environment. Different procedures have been developed for PEF ablation, e.g., pulsed-field ablation, direct current ablation, electrochemical treatment, and electroporation, each with nuanced variations of methodology and characteristics.

Peripheral arterial disease (PAD) is a chronic atherosclerotic condition where plaque accumulation can narrow and obstruct peripheral blood vessels, particularly those supplying the lower extremities. As arterial flow becomes compromised, patients can develop claudication, reduced perfusion, tissue ischemia, and even limb-threatening complications. Endothelial dysfunction and inflammatory signaling can contribute to disease progression and exacerbate vascular injury after interventional procedures.

Current treatment methods can include endovascular interventions, e.g., balloon angioplasty, atherectomy, or stent implantation, which can injure the vascular wall and trigger a healing response (e.g., smooth muscle cell proliferation, maladaptive remodeling). The healing response can lead to neointimal hyperplasia (NIH), a primary driver of restenosis that compromises vessel patency and necessitates repeat interventions.

There is a benefit to improving the pulse electric field systems and methods, e.g., for treating PAD and other vascular diseases.

### SUMMARY

An exemplary pulsed electric field system and method are provided that employs conductivity-controlled liquid medium in the moving flow of a blood vessel to induce a PEF target tissue response (e.g., reversible electroporation, pulsed field ablation, ablation of vascular plaque or atherosclerotic lesions, vascular smooth muscle cell ablation, endothelial cell stimulation, or reduction of neointimal hyperplasia). Electroporation employs brief electrical pulses to temporarily increase the permeability of cell membranes, allowing the introduction of molecules (such as DNA or drugs) into cells. The conductivity-controlled liquid medium allows more uniform electrical pulses to be generated with fewer localized or focal bursts of pulses, which can reduce the required power rating of the electric generator for the pulses and reduce the likelihood of tissue damage via more uniform targeted operation.

The exemplary flow-based pulsed electric field device may be optimized for clinical usage with a flow of conductivity-controlled liquid medium in a blood vessel to deposit energy in the entire circumference, surface, or region of the blood vessel into a predictable depth or tissue layer. The controllability of the deposition of energy facilitates and can be used in combination with a variety of treatments (therapeutic, gene, cell impermeable agent, or other biological intervention therapy described or referenced herein).

The control of depth, particularly the superficial layer, and the application across a large or whole area of the blood vessel, provides a new approach for cancer treatment. In particular, the exemplary pulsed electric field system and method can be used to deposit electrical energy across the blood vessel to exfoliate the entire or desired portion of the endothelial layer or to remove atherosclerotic or vascular plaque.

The exemplary pulsed electric field system and method can be tailored to selectively target a response in certain cells (e.g., cancer cells) while not affecting other cells (e.g., T-cells, macrophages, etc.).

The exemplary pulsed electrical field device may include features to address technical challenges associated with moving volume surface energy deposition. Increasing the number of electrodes facilitates the distribution of energy to the tissue, but at a tradeoff of other technical issues, e.g., device complexity, uneven electrical distribution that can lead to localized heating. The conductivity-controlled liquid medium mediates the electrical energy emissions and the nearby or contacting tissue to lessen or reduce focalized electrical emission and, thus, associated focalized thermal effects, reduce the need to have a large number of electrodes, provide a medium for cooling/thermal regulation, and provide a medium for secondary or tertiary control via therapeutic, gene, or other biological intervention therapy. The exemplary pulsed electric field system and method can operate without reliance on thermal effects. The exemplary pulsed electric field system and method can operate in combination with thermal effects.

The infusion and displacement of blood by the introduction of the conductivity-controlled liquid medium can also assist with the prevention of hemolysis. Hemolysis is the breakdown of red blood cells (RBCs). Intravascular hemolysis often occurs within blood vessels, e.g., due to mechanical damage (e.g., from prosthetic heart valves), infections (e.g., malaria), and immune reactions.

In addition to treatments, e.g., cancer treatments, the exemplary pulsed electrical field device can be alternatively used for cell stimulation, reversible electroporation, ablation of vascular plaque or atherosclerotic lesions, vascular smooth muscle cell ablation, endothelial cell stimulation, reduction of neointimal hyperplasia, among others.

In an aspect, a system for intravascular pulsed electric field treatment is disclosed comprising: an intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter (e.g., optionally being self-expanding or mechanically adjustable) without occluding the blood vessel, and wherein the expandable electrode assembly is configured to be deployed within the blood vessel such that the electrodes are positioned in proximity to or in contact with the vessel wall; and at least one fluid delivery lumen, including a fluid delivery lumen, configured to be positionable into the blood vessel concurrent with the intravascular catheter without occluding the blood vessel to introduce a conductive fluid into the blood vessel; and a controller coupled to (i) the at least one fluid delivery lumen and the (ii) the plurality of electrodes, wherein the controller is configured to direct conductive fluid (e.g., via control of a pump or flow/pressure regulator) to the at least one fluid delivery lumen to partially or fully displace blood at a treatment site and establish a localized conductive environment, wherein the controller is configured to direct delivery of a plurality of electrical pulse waveforms within the conductive fluid environment to direct the plurality of electrical pulse waveforms to a tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target tissue response (e.g., reversible electroporation, pulsed field ablation, ablation of vascular plaque or atherosclerotic lesions, vascular smooth muscle cell ablation, endothelial cell stimulation, or reduction of neointimal hyperplasia, or reduction of neointimal hyperplasia), and wherein the controller stores in memory a set of selectable waveforms (e.g., square, exponential, or user-defined shaped pulses) that are selectable by a user to direct delivery of the plurality of electrical pulse waveforms to induce the target tissue response.

In some embodiments, the fluid delivery lumen is integrated within the intravascular catheter as a multi-lumen catheter.

In some embodiments, the system described herein further comprises: a second catheter configured to be positionable into the blood vessel concurrent with the intravascular catheter without occluding the blood vessel to introduce a second conductive fluid into the blood vessel, wherein the controller is configured to direct conductive fluid (e.g., via control of a pump or flow/pressure regulator) to the second catheter, wherein delivery of the conductive fluid by the fluid delivery lumen and the second catheter partially or fully displaces blood at the treatment site and establishes the localized conductive environment (e.g., to prevent intravascular hemolysis).

In some embodiments, the intravascular catheter is configured with a catheter arrangement selected from the group consisting of a coaxial configuration, a concentric configuration, a parallel configuration, and an independently navigable configuration.

In some embodiments, the plurality of electrodes is configured as an electrode assembly of symmetrically arranged electrodes (e.g., respectively sized for vessel diameters in a range of 1 mm-20 mm) (e.g., wherein electrodes operate in: contact (e.g., 0-0.5 mm), non-contact (e.g., 0.5-5 mm), or hybrid modes) (e.g., wherein the plurality of electrodes are fully exposed, partially insulated, or directionally exposed) (e.g., wherein the electrodes are shaped from the group consisting of linear, curved, circumferential, or helical).

In some embodiments, the controller is configured and selectable to direct delivery of the plurality of electrical pulse waveforms to all of the plurality of electrodes simultaneously (e.g., wherein each of the plurality of electrical pulse waveforms is the same).

In some embodiments, the controller is configured and selectable to direct delivery of the plurality of electrical pulse waveforms to each of the plurality of electrodes in a pre-defined sequence (e.g., rotationally).

In some embodiments, the plurality of electrodes of the intravascular catheter are self-expanding upon being deployed from the intravascular catheter.

In some embodiments, the intravascular catheter is coupled to a handle, the handle having a manual actuator coupled to a mechanical connection to the plurality of electrodes, the manual actuator being adjustable to mechanically adjust a deployment size of the plurality of electrodes (e.g., to match a desired treatment length and diameter within a given vessel).

In some embodiments, the intravascular catheter is configured to stow the plurality of electrodes, including a central shaft electrode, in a stowed central shaft electrode configuration and to deploy the plurality of electrodes, including the central shaft electrode, in a deployed central shaft electrode configuration.

In some embodiments, the intravascular catheter is coupled to an expandable basket, wherein the intravascular catheter is configured to stow the plurality of electrodes in a stowed central shaft electrode configuration and to deploy the plurality of electrodes in a deployed central shaft electrode configuration, and wherein the plurality of electrodes are extendable within an interior region defined by the expandable basket in an expandable basket electrode configuration (e.g., wherein the plurality of electrodes, e.g., central shaft electrodes or wall electrodes, are coaxially aligned with a catheter axis, centered within a basket electrode array, and/or extendable partially or fully along a treatment region) (e.g., wherein the plurality of electrodes as central shaft electrodes have a collective diameter in a range of about 0.1 mm-2 mm) (e.g., wherein the plurality of electrodes, e.g., central shaft electrode, are fully conductive, partially insulated, or directionally exposed along selected regions) (e.g., wherein the plurality of electrodes include a central shaft electrode, conductive electrodes, a structural support element, and/or a fluid delivery conduit (lumen-integrated design)) (e.g., wherein the plurality of electrodes as an expandable electrode assembly includes 4-12 electrodes, arranged in a basket configuration adapted to conform to vessel diameters of about 1 mm-20 mm) (e.g., wherein the plurality of electrodes are symmetrically or asymmetrically distributed, linear, curved, helical, or circumferential, and self-expanding or mechanically actuated).

In some embodiments, the controller is coupled to a plurality of monopolar electrodes as the plurality of electrodes in a monopolar configuration (e.g., wherein a central shaft electrode is electrically activated in combination with the expandable electrodes in multiple configurations) (e.g., wherein the central shaft electrode and/or basket electrodes act as active electrodes, a remote return electrode (e.g., grounding pad) completes the system, and/or all basket electrodes may be: activated simultaneously, or grouped for staged activation).

In some embodiments, the controller is coupled to a plurality of radial field electrodes as the plurality of electrodes in a radial field configuration (e.g., wherein a central shaft electrode acts as positive (anode) or negative (cathode) with the expandable electrodes).

In some embodiments, the controller is coupled to a plurality of radial field electrodes as the plurality of electrodes in a radial field configuration, wherein surrounding basket electrodes of the plurality of electrodes act with opposite polarity to generate a radially distributed electric field (e.g., extending from a center outward, or from the vessel wall inward).

In some embodiments, the controller is coupled to a plurality of bipolar configuration electrodes as the plurality of electrodes in a bipolar configuration, wherein adjacent or alternating basket electrodes of the plurality of electrodes (e.g., act as positive and negative pairs, e.g., wherein a central shaft electrode is inactive, floating, or used for sensing/feedback).

In some embodiments, the controller is coupled to the plurality of hybrid configuration electrodes in a hybrid configuration (e.g., a combination of central shaft activation and inter-electrode (basket) activation, enabling localized high-field regions, and global field shaping).

In some embodiments, the system described herein further comprises: an imaging system (e.g., Optical Coherence Tomography (OCT) imaging system) configured to perform intravascular imaging (e.g., within a blood-cleared region before, during, or after delivery of electrical pulses) (wherein the conductive fluid delivery creates a localized blood-cleared region within the vessel) (e.g., wherein the controller is configured to adjust pulse, electrode position, and/or fluid parameters in real time based on sensor feedback, optionally integrating OCT or other imaging modalities).

In some embodiments, the system described herein further comprises: a sensor (e.g., an intravascular sensor in the catheter, blood vessel, or system sensor), wherein the controller is configured to adjust pulse, electrode position, and/or fluid parameters in real time based on sensor feedback.

In another aspect, a method is disclosed comprising: deploying an intravascular catheter at least one fluid delivery lumen in a blood vessel, wherein the intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter (e.g., optionally being self-expanding or mechanically adjustable) without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment; and directing delivery of a plurality of electrical pulse waveforms within the conductive fluid environment via the controller to direct the plurality of electrical pulse waveforms to a tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target tissue response (e.g., reversible electroporation, pulsed field ablation, ablation of vascular plaque or atherosclerotic lesions, vascular smooth muscle cell ablation, endothelial cell stimulation, or reduction of neointimal hyperplasia), and wherein the controller stores in memory a set of selectable waveforms (e.g., square, exponential, or user-defined shaped pulses) that are selectable by a user to direct delivery of the plurality of electrical pulse waveforms to induce the target tissue response.

In some embodiments, the plurality of electrodes is deployed in a non-contact or a hybrid mode for electroporation, pulsed field ablation, vascular smooth muscle cell modulation, or reduction of neointimal hyperplasia as the target tissue response.

In some embodiments, the plurality of electrodes is deployed in contact mode to treat plaque or when there are vessel irregularities.

In some embodiments, the method described herein further comprises: imaging the vessel wall to guide electrode positioning or adjustment of pulse and fluid delivery parameters during delivery of the plurality of electrical pulse waveforms.

In another aspect, a method for electroporation is disclosed comprising: deploying an intravascular catheter (e.g., of any of the above-discussed systems) at least one fluid delivery lumen in a blood vessel, wherein the intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment; and directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target electroporation response, and wherein the controller stores in memory a set of selectable waveforms (e.g., square, exponential, or user-defined shaped pulses) that are selectable by a user to direct delivery of the plurality of electrical pulse waveforms to induce the target electroporation response.

In another aspect, a method for pulsed field ablation is disclosed comprising: deploying an intravascular catheter (e.g., of any of the above-discussed systems) at least one fluid delivery lumen in a blood vessel, wherein the intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment; and directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target pulsed field ablation response, and wherein the controller stores in memory a set of selectable waveforms (e.g., square, exponential, or user-defined shaped pulses) that are selectable by a user to direct delivery of the plurality of electrical pulse waveforms to induce the target pulsed field ablation response.

In another aspect, a method for vascular smooth muscle cell ablation is disclosed comprising: deploying an intravascular catheter (e.g., of any of the above-discussed systems) at least one fluid delivery lumen in a blood vessel, wherein the intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment; and directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target vascular smooth muscle cell ablation response, and wherein the controller stores in memory a set of selectable waveforms (e.g., square, exponential, or user-defined shaped pulses) that are selectable by a user to direct delivery of the plurality of electrical pulse waveforms to induce the target vascular smooth muscle cell ablation response.

In yet another aspect, a method for a reduction of neointimal hyperplasia is disclosed comprising: deploying an intravascular catheter (e.g., of any one of the above-discussed systems) at least one fluid delivery lumen in a blood vessel, wherein the intravascular catheter including an expandable electrode assembly having a plurality of electrodes arranged in a basket configuration, wherein the plurality of electrodes includes 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment; and directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reduction of neointimal hyperplasia response, and wherein the controller stores in memory a set of selectable waveforms (e.g., square, exponential, or user-defined shaped pulses) that are selectable by a user to direct delivery of the plurality of electrical pulse waveforms to induce the target reduction of neointimal hyperplasia response.

In another aspect, a method is disclosed for treating atherosclerotic or vascular plaque comprising: deploying an intravascular catheter (e.g., of any one of the above-discussed systems) at least one fluid delivery lumen in a blood vessel, wherein the intravascular catheter comprising an expandable electrode assembly having a plurality of electrodes arranged in a basket configuration, wherein the plurality of electrodes comprises 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and wherein the at least one fluid delivery lumen includes a fluid delivery lumen and is configured to be positionable into the blood vessel concurrent with the intravascular catheter without occluding the blood vessel to introduce a conductive fluid into the blood vessel; and establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment, directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reduction of atherosclerotic or vascular plaque response, and wherein the controller stores in memory a set of selectable waveforms (e.g., square, exponential, or user-defined shaped pulses) that are selectable by a user to direct delivery of the plurality of electrical pulse waveforms to induce the target reduction of atherosclerotic or vascular plaque response.

### BRIEF DESCRIPTION OF DRAWINGS

FIGS. 1A-1D show an example pulsed electric field system that can uniformly emit electrical pulses via the use of an expandable electrode catheter and a conductivity-controlled liquid medium to penetrate a pre-defined depth of a target tissue for a whole or region of a hollow or tubular organ in accordance with an illustrative embodiment.

FIGS. 2A-2D show example instrumentation implementation and electrode configurations for the pulsed electric field system of FIG. 1A-1D in accordance with an illustrative embodiment.

FIG. 2E shows example geometric configurations of the electrodes.

FIGS. 3A-3E show different mechanical configurations for the pulsed electric field device of FIGS. 1A-1D in accordance with various embodiments.

FIGS. 4A-4E show example method 400 for the pulsed electric field application in accordance with various embodiments.

FIGS. 5A-5F shows a pulsed electric field methodology and computation simulation results of modeling to design a pulsed electric field device.

FIG. 6A shows the effects of an intravascular pulsed electric field on endothelial cells.

FIG. 6B shows a non-occlusive basket catheter configured for endovascular pulsed electric field (PEF) delivery to arteries within an object.

FIG. 6C shows the effect of fluid flushing on thrombus prevention during pulsed-field ablation with the catheter.

FIG. 6D shows FEA simulations to analyze the effects of pulsed-field ablation in a blood vessel.

FIG. 6E shows a non-occlusive basket catheter having a self-expandable basket electrode.

FIG. 6F shows angiographic imaging of the pulsed-field ablation.

FIG. 6G shows a histological analysis of smooth muscle remodeling and endothelial regeneration after the pulsed-field ablation.

FIG. 7 shows an example experiment and pulsed electric field system of FIG. 1A configured with an expandable electrode catheter and a flushing catheter.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference were individually incorporated by reference.

### Definition

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g., desired antioxidant release rate or viscoelasticity. The specific level in terms of wt % in a composition required as an effective amount will depend upon a variety of factors, including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition can also be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single-dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art, however, that a patient may insist upon a lower dose or tolerable dose for medical reasons, psychological reasons, or for virtually any other reason.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied, for example, by increasing or decreasing the amount of a disclosed compound and/or pharmaceutical composition, by changing the disclosed compound and/or pharmaceutical composition administered, by changing the route of administration, by changing the dosage timing, and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing the onset or initiation of a disease or condition.

As used herein, the term “prevent” or “preventing” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed.

As used herein, the terms “optional” or “optionally” mean that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom, or condition thereof. The effect can be therapeutic in terms of a partial or complete cure of a disease, condition, symptom, or adverse effect attributed to the disease, disorder, or condition. The term “treatment” as used herein can include any treatment of a disease disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder, or condition, e.g., impeding its progress; and relieving the disease, disorder, or condition, e.g., causing regression of the disease, disorder, and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such an agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing the rate of advancement of a disease, disorder, condition, or side effect.

### Example System

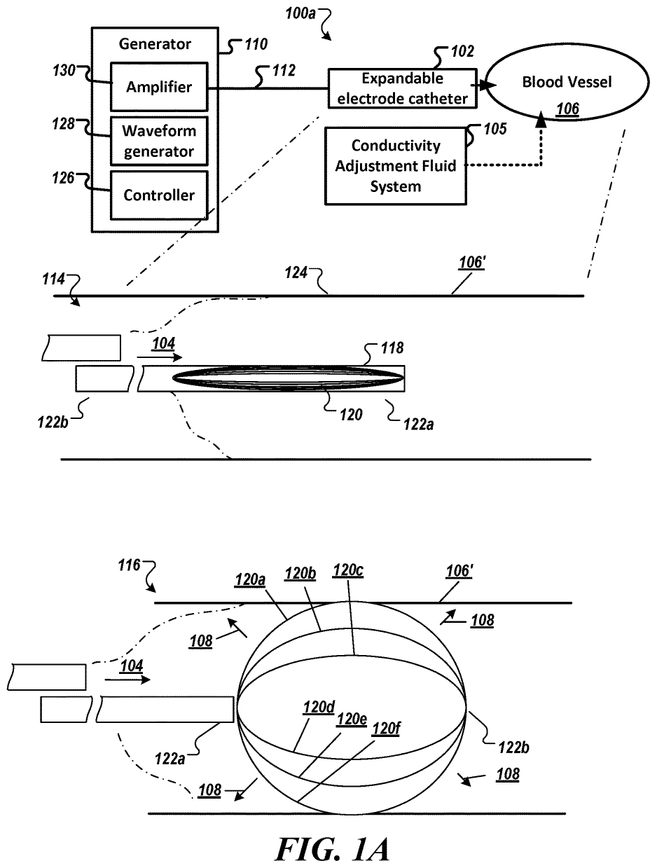

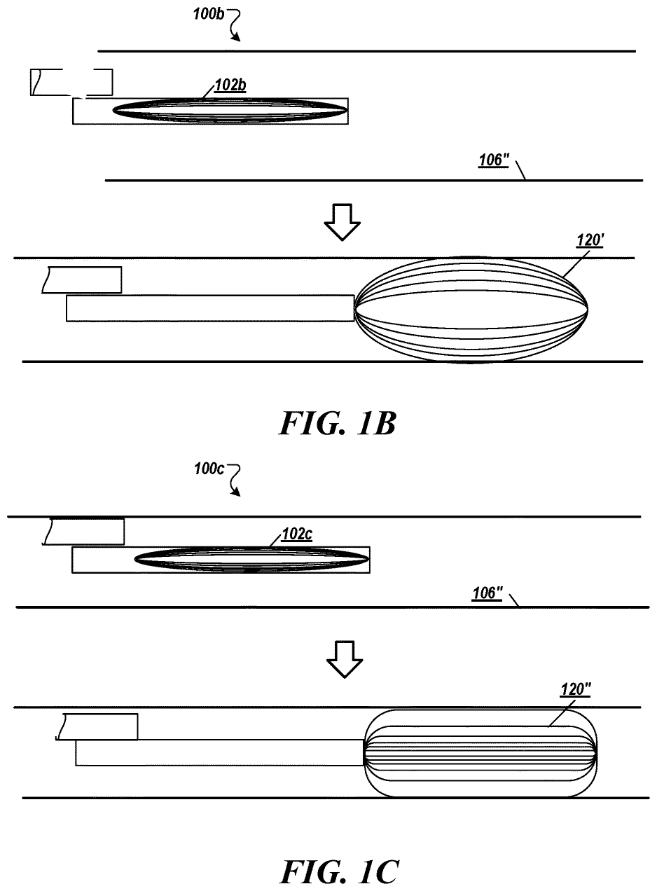

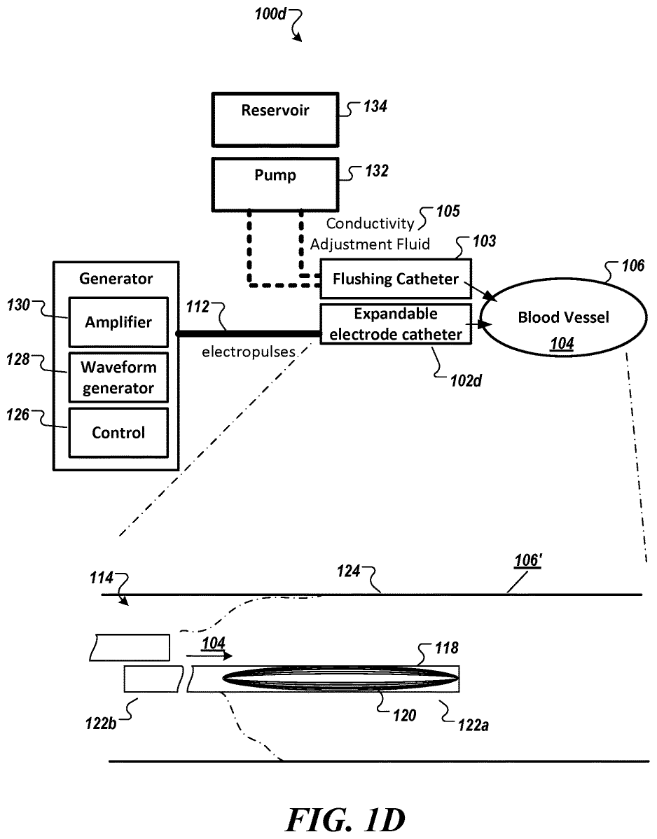

FIG. 1A shows an example pulsed electric field system 100 (shown as 100a, 100b, 100c, 100d, 100e) that can uniformly emit electrical pulses via the use of an intravascular catheter 102 and a conductivity-controlled liquid medium 104 to penetrate a pre-defined depth of a target tissue for a whole or region of a blood vessel 106 in accordance with an illustrative embodiment. The pulsed electric field 108 induces a tissue response to invoke, depending on the electrical characteristics, reversible electroporation, pulsed field ablation, ablation of vascular plaque or atherosclerotic lesions, vascular smooth muscle cell ablation, endothelial cell stimulation, or reduction of neointimal hyperplasia, among others. Reversible electroporation employs brief electrical pulses to temporarily increase the permeability of cell membranes, allowing the introduction of molecules (such as DNA or drugs) into cells. Pulsed Field Ablation (PFA) is a non-thermal treatment for atrial fibrillation (AFib) that uses short, high-energy electrical pulses (reversible electroporation) to destroy heart tissue, causing arrhythmia. Vascular smooth muscle cell (VSMC) modulation is the phenotypic switching of cells from a quiescent, contractile state to a synthetic, proliferative state in response to vascular injury, inflammation, or mechanical stress. This high plasticity causes VSMCs to lose marker proteins (e.g., SM-MHC) and take on macrophage- or fibroblast-like roles, contributing to atherosclerosis and restenosis. Reduction of neointimal hyperplasia is to inhibit, via pulsed electric field application, the proliferation and migration of vascular smooth muscle cells and promote rapid re-endothelialization of the injured vessel.

The intravascular catheter 102 may include an expandable electrode assembly having a plurality of electrodes arranged in a basket configuration, where the plurality of electrodes comprises 4-12 electrodes, each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

One or more conductivity-controlled liquid medium 104 are introduced into the blood vessel by at least one fluid delivery lumen that includes a fluid delivery lumen configured to be positionable into the blood vessel 106 concurrent with the intravascular catheter without occluding the blood vessel to introduce a conductive fluid into the blood vessel to establish at a target site in the blood vessel to allow more uniform electrical pulses 108 to be generated with less localized or focal bursts of pulses, which can reduce the required power rating of the electric generator for the pulses, as well as reduce heating effects. The electroporation/ablation can rapidly eliminate all the endothelium at the target site in the vessel and in a non-invasive mechanical manner.

In the example shown in FIG. 1A, the intravascular catheter 102 operates with a generator 110 through a cable 112. The intravascular catheter 102 is configured to be inserted into the blood vessel 106 (shown as 106′) while in the stowed configuration 114 and then deployed in the blood vessel 106′ to the deployed configuration 116.

The intravascular catheter 102 includes a tubular member 118 and a plurality of electrodes 120. The tubular member 118 has a first end 122a and a second end 122b. The plurality of electrodes 120 includes at least 4 electrodes (e.g., shown as 120a, 120b, 120c, . . . 120n) that is housed in the first end 122a of the tubular member 118 when in the deployed configuration 114 and is configured to extend out of the tubular member 118, via movement of the tubular member or movement of the plurality of electrodes, to be in the deployed configuration 116. During deployment, the electrodes 120 extend in different angular directions to each other to come into proximity or contact with the surface tissue 124 of the blood vessel 106, e.g., to fill the shape of the blood vessel 106. The conductivity-controlled liquid medium 104 is a natural or synthetic liquid introduced into the blood vessel 106.

The electrical system may operate the electrodes in monopolar, bipolar, or multipolar configurations. The pulses may be 50V-3000 V, 5 ns-1000 μs, 1-1000 pulses, 0.1-10 Hz, and/or selectable waveform including square, exponential, monophasic, biphasic, or custom-shaped pulses. The penetration depth may be controlled via pulse, conductivity, and geometry.

The treatment length may be between 1 mm and 10 mm, preferably 2-5 mm.

The intravascular catheter 102 may include an expandable structure to reduce fluid volume required.

Conductivity-controlled liquid medium and adjustments. The conductivity-controlled liquid medium 104 has a defined conductance or resistance property and can be adjusted to a conductivity ratio by the introduction of a conductivity adjustment fluid 105. The conductivity ratio of the conductivity-controlled liquid medium 104 may be established (i) between 1 and 10 between the liquid medium 104 in the blood vessel 106 and the surface tissue 124 when the electrodes 120 are intended to contact the target tissue (e.g., surface tissue) during the delivery of the electropulses and (ii) between 0.1 and 0.5 when the electrodes 120 are intended to not contact the target tissue. The conductivity-controlled liquid medium 104 may include calcium, bleomycin, cisplatin, or other cell impermeable agent introduced by the conductivity adjustment fluid 105. In some embodiments, the conductivity-controlled liquid medium 104 is delivered at a flow rate in the range of about 1 mL/s-30 mL/s, and preferably about 2 mL/s-15 mL/s, corresponding to approximately 1.0×-3.0× the native blood flow rate (e.g., native arterial flow of about 2 mL/s-15 mL/s), such that the delivered fluid is capable of overcoming incoming flow and maintaining a transient blood-cleared or blood-reduced zone at the treatment site.

In some embodiments, the conductivity-controlled liquid medium 104 has an established conductivity ratio (for contactless pulsed electric field application with the nearby tissue) between a value of 0.1-0.2, 0.2-0.3, 0.3-0.4, 0.4-0.5, 0.1-0.3, 0.2-0.4, 0.3-0.5.

Table 1 shows example conductivity ratios to be employed for the pulsed electric field operation.

TABLE 1

| | |
|---|---|
| Contact | Conductivity ratio between 0.1 and 10 between the liquid |
| | medium in the hollow or tubular organ and the target tissue |
| | (e.g., surface tissue) of the hollow or tubular organ |
| Non-Contact | Conductivity ratio between 0.1 and 10 between the liquid |
| | medium in the blood vessel and the endothelium |

The electrodes 120 are configured to uniformly, directly or indirectly, emit the plurality of electrical pulses 108 in the liquid medium 104 (e.g., while in contact with the tissue or only through the liquid medium) to penetrate at least a pre-defined depth into the surface tissue 124 as a pulsed electric field application to induce a tissue response of the target tissue of the hollow or tubular organ. The number of electrodes 120 can be 4, 5, 6, 7, 8, 9, 10, 11, or 12, depending on the size of the vessel 106 (e.g., vessel diameters in the range of 1-20 mm).

In some embodiments, the electrodes 120 comprise 4-12 symmetrically arranged electrodes. The electrodes may operate in: contact mode (0-0.5 mm), non-contact mode (0.5-5 mm), or hybrid modes. The electrodes 120 may be fully exposed, partially insulated, or directionally exposed. The electrodes may be energized simultaneously, sequentially, or rotationally. The electrodes may be optionally self-expanding or mechanically adjustable. The electrodes 120 may have shapes including linear, curved, circumferential, or helical. In non-contact or hybrid modes, the energy is delivered effectively in regions where direct electrode contact is limited due to plaque presence or vessel irregularities.

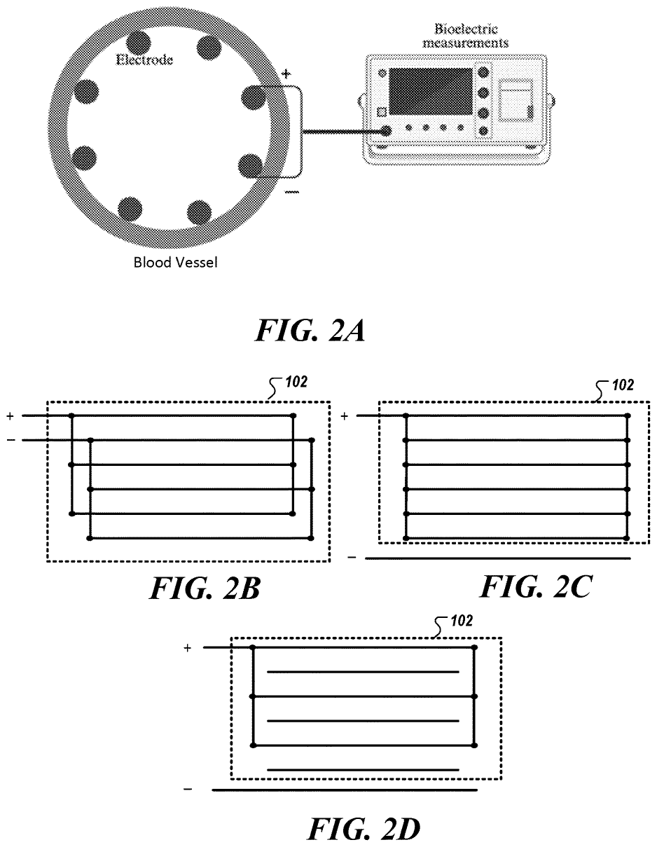

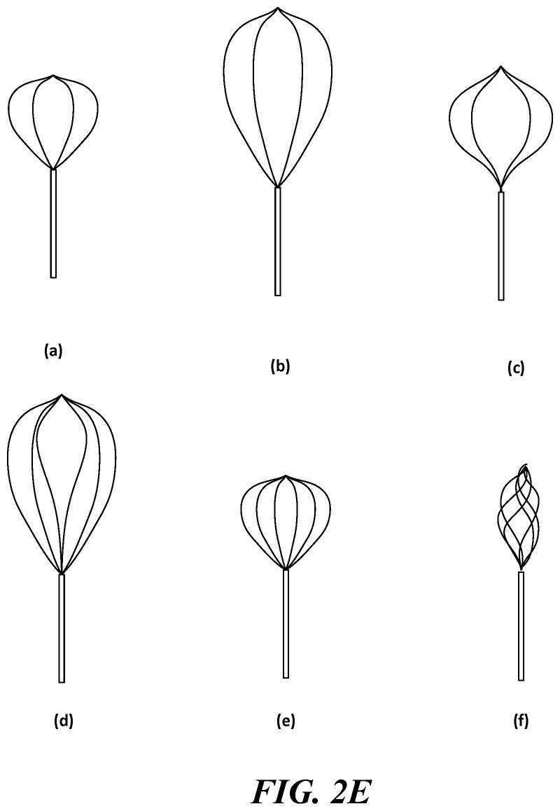

In some embodiments, the electrodes 120 may have between 4 and 12 electrode members, each configured to flexibly extend to contact or to come proximal to the vessel wall. The electrodes 120 may be made of a material typically used for ablation or surgical electrodes, e.g., stainless steel, titanium, nitinol. The electrodes 120, when expanded, can form a basket configured to conform to the shape of the blood vessel (e.g., see FIG. 2B, 3A, 3D, 3E).

The desired depth can be up to 10 mm of the vascular cells and tissues or from the electrode catheter tip (e.g., for perivascular tumor ablation).

Generator. The generator 110 is an electrical instrument configured, via a control program for a pulsed electric field application (e.g., electroporation, reversible electroporation, pulsed field ablation, or targeted cell stimulation), to generate a monophasic electro pulse having a square wave, to deliver the electrical pulses to the electrodes 120. In FIG. 1A, the generator 110 is configured with a controller 126, a waveform generator 128, and an amplifier 130. The electrical instrument is configured, via a control program of the controller, to generate a set of monophasic or biphasic electropulses, e.g., per Table 2, for the reversible electroporation and subsequent exfoliation of the surface tissue, electroporation, pulsed field ablation, and cell stimulation.

TABLE 2

| | Generator Parameters |
|---|---|
| Monophasic | Voltage range: 50 V-3000 V, Pulse width: 10 μs-1000 μs |
| | (e.g., 50-150 μs), Interpulse delay: 0.1 μs-10 s |
| | (triggerable via external biosignal, e.g., muscle contraction, |
| | nerve excitation, or cardiac rhythm), Pre-defined number |
| | of electrical pulses: minimum 3-8 pulses, maximum |
| | 10,000 pulses |
| Biphasic | Voltage range: 50 V-3000 V, Pulse width per phase: 5 |
| (symmetric | ns-100 μs, Inter-phase delay: 1 μs-10 μs, Interpulse |
| or | delay: 0.1 μs-10 s (triggerable via external biosignal, |
| asymmetric) | e.g., muscle contraction, nerve excitation, or cardiac |
| | rhythm), Pre-defined number of electrical pulses: |
| | minimum 3-8 pulses, maximum 10,000 pulses, Burst |
| | mode, 1-1000 bursts, each burst comprising 1-1000 |
| | electrical pulses, Inter-burst delay: 0.1 s-10 s |

The electrical instrument (e.g., 110) may be configured, via a control program, to generate electropulses having a selectable interpulse delay (e.g., triggerable via an external biosignal, e.g., muscle contraction, nerve excitation, or cardiac rhythm). The control program may have pre-defined output configurations for reversible electroporation and subsequent exfoliation of the surface tissue, electroporation, pulsed field ablation, cell stimulation, etc.

The electrical instrument (e.g., 110) may be configured, via a control program for reversible electroporation, to generate biphasic electropulses comprising symmetric or asymmetric square waves.

The electrical instrument (e.g., 110) may be configured, via the control program, to generate electropulses comprising a 0.1 μs-10 s interpulse delay.

The electrical instrument (e.g., 110) may be configured, via the control program, to deliver a pre-defined number of electropulses (e.g., having a minimum of 3-8 electropulses and a maximum of 10,000 electropulses).

FIGS. 1B and 1C show the intravascular catheter 102 (shown as 102b, 102c) in a blood vessel 106 (shown as 106″). FIG. 1B shows the electrodes 120 (shown as 120′) having a curved profile. FIG. 1C shows the electrodes 120 (shown as 120″) having an elongated or rectangular profile.

FIG. 1D shows an example pulsed electric field system 100d configured with an intravascular electrode catheter 102 (shown as 102d) configured to operate with a conductivity adjustment liquid introducer 103 (shown as “flushing catheter” 103). The flushing catheter 103 provides for the introduction of the liquid 105, as a portion of the liquid medium 104, to be dispensed during the pulsed electric field application. The flushing catheter 103 is coupled to a pump 132to actively introduce or recirculate a portion of the liquid medium (104) during the pulsed electric field application.

Imaging and Feedback. The system may operate with imaging and feedback. The conductive fluid delivery may be configured to create a localized blood-cleared region within the vessel, and wherein the system further comprises an Optical Coherence Tomography (OCT) imaging system configured to perform intravascular imaging within the blood-cleared region before, during, or after delivery of electrical pulses. Sensors measuring impedance, conductivity, or TEER, may be used to optionally perform impedance tomography. The controller may be configured to adjust pulse, electrode, and fluid parameters in real time based on sensor feedback, optionally integrating OCT or other imaging modalities. Imaging data may be used to assess plaque morphology and guide electrode positioning or adjustment of pulse and fluid delivery parameters.

Adjacent electrodes (120) may be used to measure the electrical properties of the vessel wall or surrounding tissue before, during, and after delivery of pulsed electric field energy. The measurements may include impedance, conductivity, or related electrical parameters. Such measurements enable real-time feedback control of the system, allowing dynamic adjustment of pulse parameters, electrode activation, and conductive fluid characteristics to optimize treatment outcomes.

Clinical Implications. The system may be configured for use before stenting, after restenosis, or as a standalone therapy in coronary, peripheral, carotid, and other arteries.

### Example Instrument Configuration

FIGS. 2A-2D show example instrumentation implementation and electrode configurations for the pulsed electric field system of FIG. 1A-1D in accordance with an illustrative embodiment. FIG. 2A shows an example instrumentation implementation 200. In FIG. 2A, the intravascular catheter 102 is shown coupled to the generator 110 and a bioelectrical measurement instrument 202. The bioelectrical measurement instrument 202 may be used to measure the impedance of the electrodes in the liquid medium 104 to regulate or change the conductivity ratio to the range described in relation to Table 1.

FIGS. 2B-2D shows example electrode configurations. FIG. 2B shows a bipolar basket electrode for blood vessel PEF, where the electrodes are marked positive and negative alternatively. In FIG. 2C, a monopolar electrode is shown for the blood vessel PEF with a distal return electrode, where all electrodes (e.g., 6 electrodes) are marked positive with the distal (electrode). In FIG. 2D, a partial mode of the monopolar basket electrode is shown for blood vessel PEF, where a subgroup of the electrodes is marked positive, and another subgroup is marked as negative alternatively (e.g., 3 electrodes are marked positive and negative alternatively). The number of electrodes may be between 4 and 12.

FIG. 2E shows example geometric configurations of the electrodes.

### Mechanical Deployment of Electrodes

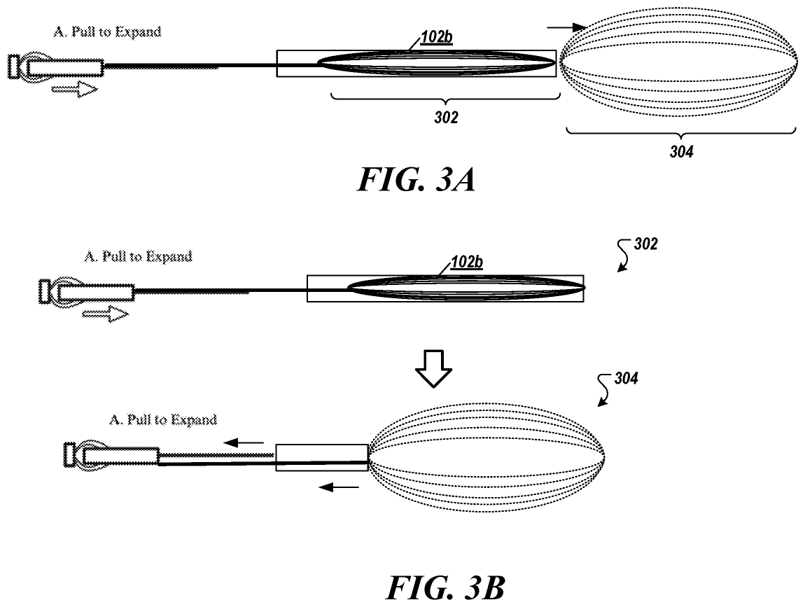

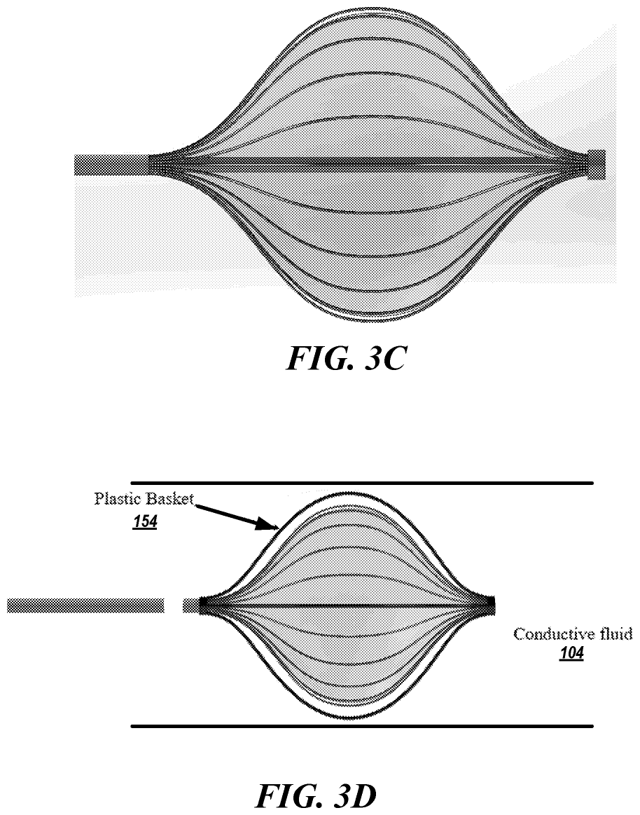

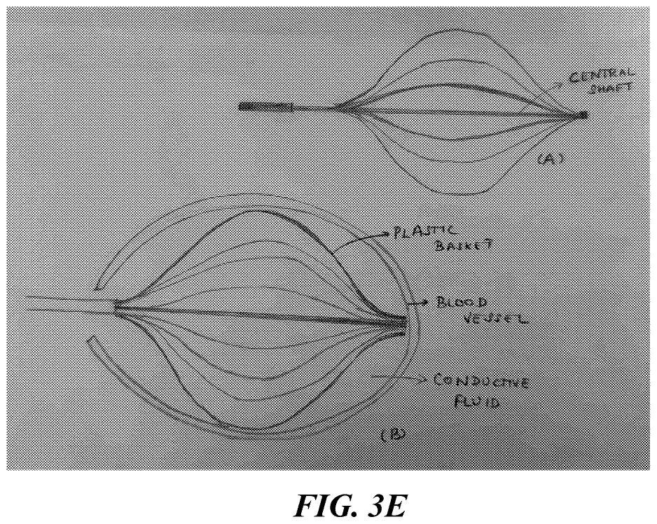

FIGS. 3A-3C show different mechanical configurations for the pulsed electric field device of FIGS. 1A-1D. In FIG. 3A, a push action by a handle causes the electrode to be deployed from the stow configuration 302 to the expanded configuration 304. The external sheath may be advanced distally (“push to expand”) to deploy the electrodes. The configuration can provide the controlled expansion and positioning of the electrodes within the blood vessel.

In FIG. 3B, the handle has two connections, one to the electrodes and a second to the surrounding sheath. A pulling action at the handle pulls the surrounding sheath back to cause the electrode to be deployed from the stow configuration 302 to the expanded configuration 304. An external sheath may be retracted proximally (“pull to expand”) to allow expansion of the self-expanding electrodes. The action may provide the electrodes to contact the inner wall of the blood vessel for a conforming geometry.

The self-expanding electrode assembly is deployed within the blood vessel in the presence of a conductive fluid. The conductive fluid surrounds the electrodes and establishes a localized conductive environment, enabling improved electrical field distribution and efficient delivery of pulsed electric field energy to the vessel wall.

In some embodiments, the expandable electrode catheter is configured with an expandable internal structure, such as a balloon or expandable support, to assist in stabilizing the device, controlling electrode spacing, and defining a treatment volume within the blood vessel. The expandable electrode catheter may adopt a conformal mechanical profile, for example, through the use of an expandable non-conductive envelope or plastic basket. The configuration may provide the electrode assembly to conform to the geometry of the target organ or vessel, to improve electrode positioning, field uniformity, and treatment consistency. The presence of the conductive fluid within the envelope-defined region further enhances uniform electric field distribution in plaque-containing vessels, particularly in regions with irregular geometry or calcified structures.

Non-conductive (Plastic Basket). FIG. 3D shows an example electrode assembly (e.g., 102) comprising an expandable non-conductive envelope (154). The assembly (e.g., 102) includes a plurality of expandable members formed of a non-conductive material (polymeric or plastic material), the envelope being stowable within the catheter and expandable to define a controlled volume surrounding the electrodes, allowing uniform conductive fluid distribution and limiting direct electrode contact in the vessel wall. In some embodiments, the electrodes expand within the envelope volume. In some embodiments, the envelope defines a controlled space between electrodes and the vessel wall. In some embodiments, the envelope is configured to reduce thermal hotspots by preventing direct electrode contact. In some embodiments, the envelope is configured to contribute to a uniform electric field and current distribution. In some embodiments, the envelope is configured to guide electrode positioning during deployment. In some embodiments, the envelope comprises a biocompatible polymeric or plastic material.

The non-conductive plastic basket configuration may have an electrode configuration, e.g., shown as described in relation to FIG. 2E. In a monopolar configuration, the electrodes may be energized relative to a distal grounding pad. In a bipolar configuration, intercalated electrodes may function as positive and negative electrodes, eliminating the need for a distal grounding pad. In a partial activation configuration, a subset of the electrodes may be selectively energized relative to the grounding pad (206), enabling localized and targeted treatment within the vessel.

FIG. 3C shows another example mechanical profile for the intravascular catheter 102, e.g., to conform to the shape of the blood vessel (e.g., 106).

FIG. 3D shows the intravascular catheter 102 configured with an internal volume balloon 152 and an external envelope guide 154.

Electrode Activation Modes. FIG. 3E, subpanel (a), shows an intravascular catheter (e.g., 102) configured with a central shaft of the basket electrode. FIG. 3E, subpanel (b) shows the plastic basket is disposed over the electrodes and placed in the blood vessel in the presence of the conductive fluid. The expandable electrode assembly may include a central shaft electrode positioned along a longitudinal axis of the catheter, extending within the interior region defined by the expandable basket electrode configuration. The central shaft electrode may be: coaxially aligned with the catheter axis, centered within the basket electrode array, and extending partially or fully along the treatment region.

The central shaft electrode may have a diameter in the range of about 0.1 mm-2 mm, and may be: fully conductive, partially insulated, or directionally exposed along selected regions.

The central shaft may function as: a dedicated electrode, a structural support element, and/or a fluid delivery conduit (lumen-integrated design). The expandable electrode assembly may comprise about 4-12 electrodes, arranged in a basket configuration adapted to conform to vessel diameters of about 1 mm-20 mm. The electrodes may be: symmetrically or asymmetrically distributed, linear, curved, helical, or circumferential, and self-expanding or mechanically actuated.

In the monopolar configuration, the central shaft electrode may be electrically activated in combination with the expandable electrodes in multiple configurations, including (i) where the central shaft electrode and/or basket electrodes act as active electrodes, where a remote return electrode (e.g., grounding pad) completes the circuit, or where all basket electrodes may be: activated simultaneously, or grouped for staged activation. In a radial field configuration, the central shaft electrode may act as positive (anode) or negative (cathode). The surrounding basket electrodes act with opposite polarity. This configuration may generate a radially distributed electric field extending outward from the center or inward from the vessel wall.

In a bipolar configuration, adjacent or alternating basket electrodes may function as positive and negative pairs, where the central shaft electrode may be inactive, floating, or used for sensing/feedback.

In the hybrid configuration, the electrode operation may be a combination of the central shaft activation and inter-electrode (basket) activation, enabling localized high-field regions and global field shaping.

In partial activation mode or sector-based control, the electrode operation may be a combination where a subset of electrodes (e.g., 2-4 of 6 electrodes) may be selectively activated to target localized plaque regions, to avoid sensitive anatomical regions, or to adapt to non-uniform vessel geometry. The central shaft electrode may be included or excluded in such activation schemes.

In sequential/rotational activation, the electrodes may be activated: sequentially, rotationally, or in predefined activation patterns, to improve coverage, reduce peak current, and enhance safety. The electrode system, including the central shaft, may operate in: contact mode (electrodes touching vessel wall, about 0-0.5 mm), non-contact mode (electrodes spaced about 0.5-5 mm from tissue), or hybrid mode. The presence of conductive fluid enables the effective field coupling in non-contact configurations and improved energy uniformity in irregular or plaque-containing vessels.

Integration with Fluid Delivery System. The central shaft may optionally include: internal lumen(s) for conductive fluid delivery, side openings for radial fluid dispersion, and/or coordinated operation with external flushing catheter(s). Electrical activation may be synchronized with fluid delivery or independently controlled. The inclusion of a central shaft electrode can provide enhanced electric field control and symmetry, improved penetration depth modulation, reduced dependence on direct electrode-tissue contact, flexibility across monopolar, bipolar, and hybrid modes, and improved treatment consistency in heterogeneous vascular environments, including plaque.

### Example Intravascular Catheter and Flushing Catheter Configuration

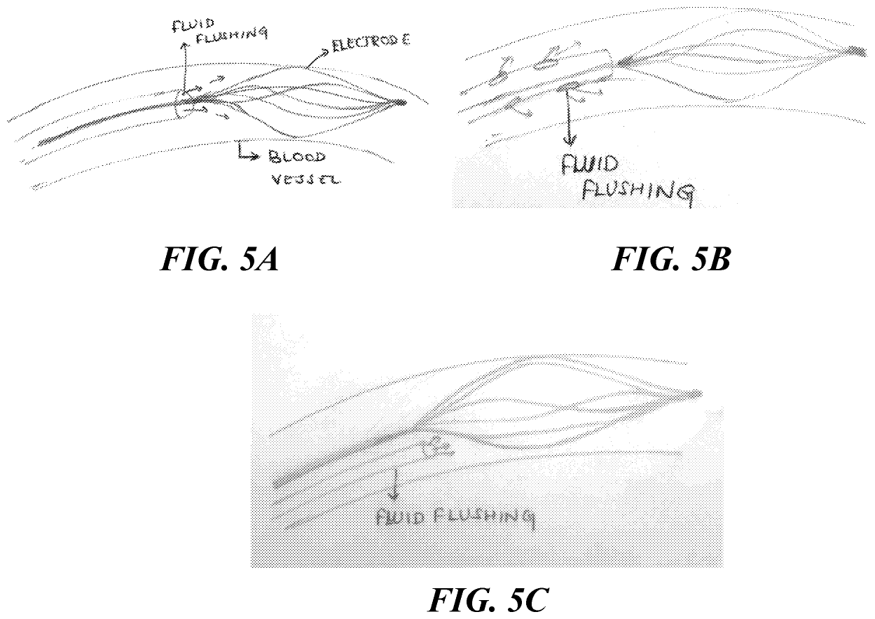

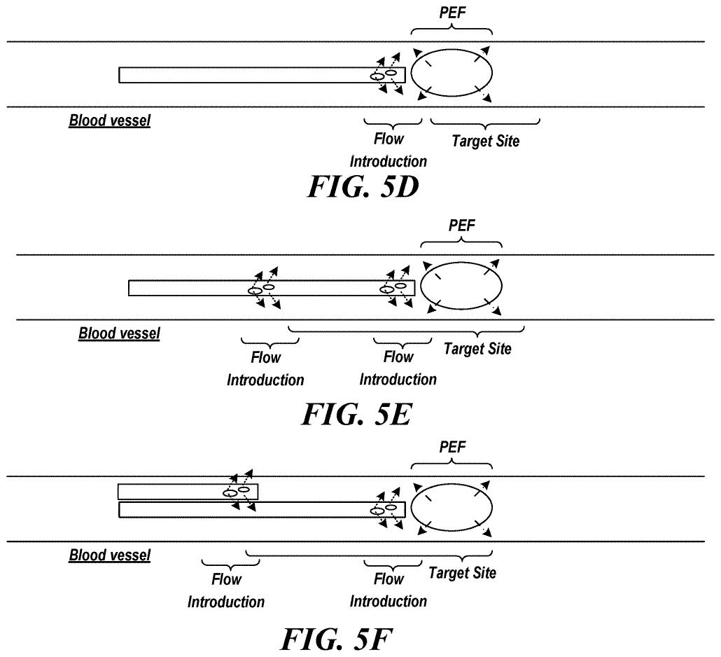

FIGS. 5A-5C show example intravascular catheter and flushing catheter configurations for an expandable basket electrode deployed within a blood vessel for modulation of the local electrical environment. In FIG. 5A, the intravascular catheter (e.g., 102) and flushing catheter (e.g., 103) are integrated to provide radially-directed fluid flushing from a proximal port to displace blood circumferentially around the electrode array.

In FIG. 5B, the intravascular catheter (e.g., 102) and flushing catheter (e.g., 103) are integrated and include multiple proximal fluid exit ports to provide distributed, circumferential flushing for more uniform blood displacement and improved electrode-tissue interface. In alternative implementations, directional flushing may be achieved by selectively activating ports on one side (e.g., right-side or left-side only) depending on plaque location.

FIG. 5C shows the intravascular catheter (e.g., 102) and flushing catheter (e.g., 103) configured for parallel (axial) fluid flushing along the catheter shaft adjacent to the electrode assembly, creating a localized low-conductivity region to enhance electroporation efficiency.

In some embodiments, the conductive fluid is configured to displace blood, form a transient conductive column, and/or establish a controlled conductive pathway. The fluid flow may be distal, proximal, radial, or axial, e.g., wherein fluid is delivered at 5-30 mL/s, e.g., where the fluid flow is 1.0×-3.0× native blood flow.

The conductive fluid may include saline, buffered electrolyte solution, or biologically compatible fluids, or combinations thereof, that maintain osmolarity and ionic strength. The fluid may be delivered at ambient or reduced (chilled) temperature. The fluid conductivity may be 0.1-3.0 S/m to establish a controlled conductive pathway for electroporation. The conductivity ratio may be 0.1-10 to establish the conductivity controlled environment. The conductive fluid may optionally include conductivity adjustment agents, therapeutic agents, or combinations thereof, including drugs, proteins, genes, or cells, without departing from vascular safety and iso-osmolar conditions, and is compatible with drug-eluting therapies.

The conductive fluid may include introduced fluid, native blood, or combinations thereof. The fluid may be delivered into the target site before, during, continuously, or synchronized with pulses (i.e., PEF operation).

The fluid delivery may be via syringe or automated pump. The fluid delivery may be synchronized with pulse delivery.

In some embodiments, the system includes fluid aspiration, recirculation, or controlled outflow. The fluid may reduce thrombus formation. The fluid may act as a thermal heat sink. The fluid may reduce power requirements and enable fewer electrodes. The conductive fluid may reduce electrical conductivity heterogeneity in plaque-containing regions to enable a more uniform electric field distribution.

Fluid Delivery Pathways and Spatial Flow Configurations. The system may be configured to deliver a conductive fluid into a blood vessel at a flow rate sufficient to partially or fully displace native blood from a localized treatment region, thereby establishing a controlled conductive environment for pulsed electric field delivery. The conductive fluid may be delivered at a flow rate in the range of about 5 mL/s-30 mL/s, and preferably about 5 mL/s-20 mL/s, corresponding to approximately 1.0×-3.0× the native blood flow rate (e.g., native arterial flow of about 2 mL/s-15 mL/s), such that the delivered fluid is capable of overcoming incoming flow and maintaining a transient blood-cleared or blood-reduced zone at the treatment site. The conductive fluid may be introduced through one or more outlet configurations, including: a distal tip outlet for forward-directed flushing, side ports along the catheter shaft, radially oriented apertures positioned within the electrode region, circumferentially distributed outlets, and/or porous or diffusive delivery regions enabling uniform dispersion.

The conductive fluid may be delivered in one or more flow directions including: axial flow along the vessel lumen, radial flow directed toward the vessel wall, distal flow toward the treatment region, proximal flow to create localized recirculation or containment of the conductive zone, circumferential or distributed flow for uniform displacement, directional flow biased toward a selected vessel region (e.g., plaque-facing side), and/or helical or swirling flow patterns to enhance mixing and displacement.

In some embodiments, the conductive fluid is delivered as a continuous flow, a pulsatile flow, or a bolus injection. In some embodiments, the fluid delivery is initiated prior to pulse delivery to establish a cleared region, maintained during pulse delivery to sustain conductivity conditions, and/or continued after pulse delivery to dissipate heat and reduce thrombogenic risk.

The fluid delivery outlet(s) may be positioned relative to the electrode assembly in one or more configurations, including co-located within the electrode region, proximally offset, or distally offset. An axial spacing between the fluid outlet and the electrode assembly may be defined in the range of approximately 0.5 mm to 20 mm, preferably 2 mm to 10 mm. In configurations comprising a separate or parallel fluid delivery catheter, a radial spacing between the fluid delivery pathway and the electrode assembly may be approximately 0.5 mm to 5 mm, preferably 1 mm to 3 mm. In certain embodiments, the spacing may be defined relative to vessel diameter, including: axial spacing of approximately 0.1× to 1× the vessel diameter, and/or radial spacing of approximately 0.05× to 0.3× the vessel diameter. The relative positioning may be selected to ensure that the delivered fluid establishes a localized region of modified conductivity overlapping at least a portion of the electric field distribution.

The conductive fluid is configured to: reduce electrical conductivity gradients caused by blood and plaque heterogeneity, improve electric field uniformity within the vessel wall, enhance energy coupling in both contact and non-contact electrode modes, reduce thrombus formation by minimizing stagnant blood regions, and act as a thermal sink to dissipate Joule heating effects.

### Example Method

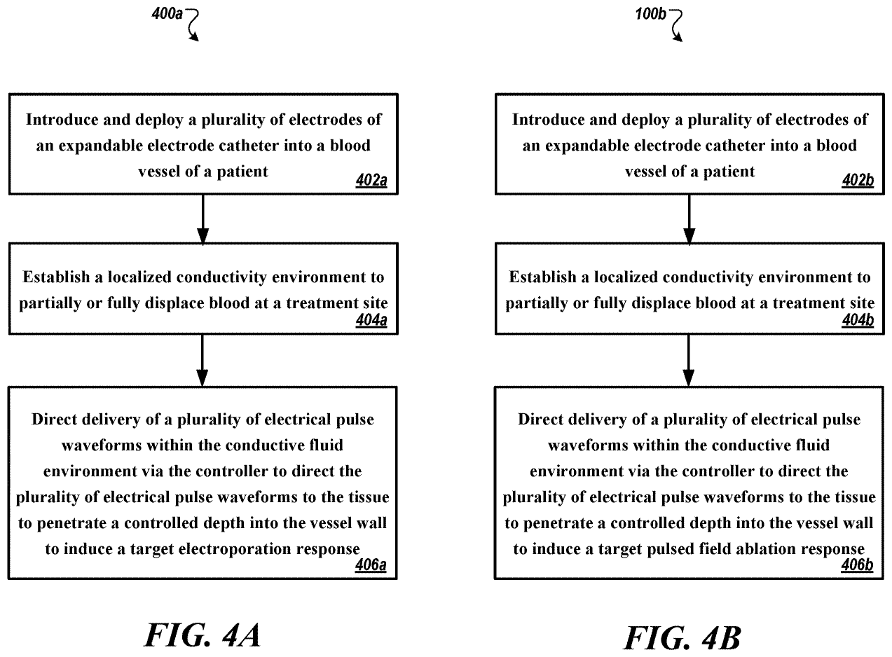

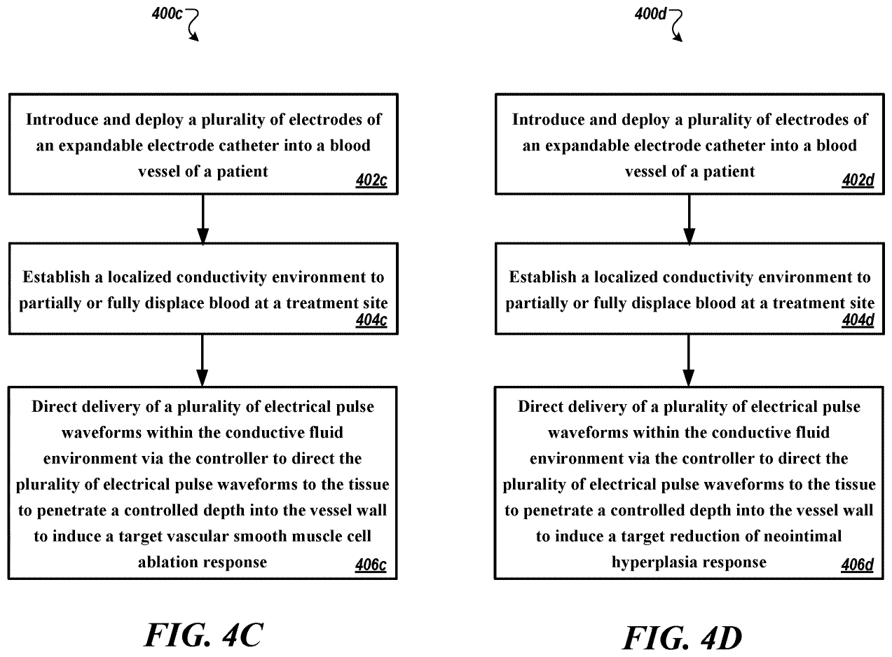

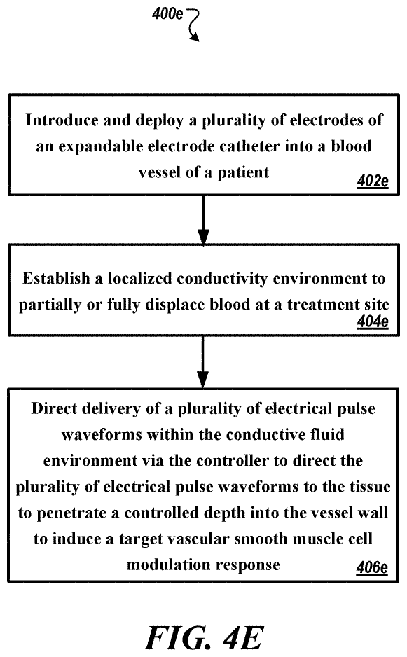

FIGS. 4A-4E show example method 400 (shown as 400a, 400b, 400c, 400d) for the blood-vessel pulsed electric field application in accordance with various embodiments. The method may include a treatment with pulsed electric field application to (i) enhance electroporation for permeabilization and molecular delivery (includes reversible electroporation, drug uptake, and gene/biologic delivery), (ii) induce endothelial cell stimulation or low-intensity electrical stimulation, (iii) induce non-thermal ablation via electroporation (includes reversible electroporation (IRE) and pulsed field ablation (PFA)), (iv) cause neointimal hyperplasia (NIH) treatment/VSMC modulation, (v) induce plaque modification (soft and mixed plaque), (vi) induce perivascular or tumor ablation, (vii) induce combined electroporation therapy (permeabilization+ablation), (viii) reduction of atherosclerotic or vascular plaque response.

Table 3 shows example properties or control settings for the pulsed electric field, along with the fluid medium flow rate.

### Table 3: Treatment Operating Configurations

TABLE 3

| Application | Example operation configuration |
|---|---|
| Electroporation for | Apply a voltage of about 50 V-1000 V, for about 1-200 pulses, |
| Permeabilization and | using monophasic or biphasic waveforms (symmetric or |
| Molecular Delivery | asymmetric), with pulse durations of about 100 ns-10 ms, to |
| (includes reversible | induce controlled and reversible membrane permeabilization and |
| electroporation, drug | facilitate intracellular delivery of therapeutic agents, drugs, or |
| uptake, and | genetic material without substantial cell death, wherein a fluid |
| gene/biologic delivery) | medium may be delivered at a flow rate of about 1-30 mL/s |
| | (preferably 2-15 mL/s), the fluid comprising a conductive fluid, |
| | therapeutic agents, drugs, genes, proteins, cells, or combinations |
| | thereof, and configured to establish a localized conductive and/or |
| | delivery environment. |
| Endothelial Cell | Apply a voltage of about 50 V-1000 V, for about 10-1000 |
| Stimulation/Low- | bursts, each burst comprising about 30-150 pulses, using |
| Intensity Electrical | monophasic or biphasic waveforms (symmetric or asymmetric), |
| Stimulation | with pulse durations of about 2 μs-10 μs per phase, and an inter- |
| | phase delay of about 1 μs-5 μs, and an inter-pulse delay of about |
| | 1 μs-10 μs, wherein an inter-burst delay of about 0.1 s-1 s is |
| | applied, to induce sub-threshold stimulation and promote |
| | endothelial cell proliferation and functional recovery without |
| | causing irreversible membrane disruption, wherein a fluid medium |
| | is optionally delivered to provide a conductive environment and/or |
| | to deliver bioactive agents that support endothelial repair. |
| Non-Thermal Ablation | Apply a voltage of about 100 V-3000 V, for about 5-1000 |
| via Electroporation | pulses, using monophasic or biphasic waveforms (symmetric or |
| (includes irreversible | asymmetric), with pulse durations of about 100 ns-100 ms, |
| electroporation (IRE) and | delivered in contact, non-contact, or hybrid modes, to induce |
| pulsed field ablation | irreversible membrane disruption and targeted cell death with |
| (PFA)) | minimal or controlled thermal effects, wherein, in vascular |
| | applications, a conductive fluid is delivered at a flow rate of about |
| | 1-30 mL/s (preferably 2-15 mL/s) to partially or substantially |
| | displace blood, establish a localized conductive pathway, and |
| | reduce thrombus formation, and wherein the fluid may optionally |
| | comprise therapeutic or biologically active agents. |
| Neointimal Hyperplasia | Apply a voltage of about 100 V-2000 V, for about 10-500 |
| (NIH) Treatment/ | pulses, using monophasic or biphasic waveforms (symmetric or |
| VSMC Modulation | asymmetric), with pulse durations of about 1 μs-10 ms, delivered |
| | in contact, non-contact, or hybrid modes, to selectively modulate |
| | or ablate vascular smooth muscle cells while preserving |
| | extracellular matrix and enabling endothelial recovery. |
| | wherein, in vascular applications, a conductive fluid is delivered at |
| | a flow rate of about 1-30 mL/s (preferably 2-15 mL/s) to |
| | partially or fully displace blood within the treatment region, reduce |
| | electrical conductivity heterogeneity, and minimize thrombus |
| | formation, and wherein the fluid may optionally comprise |
| | therapeutic agents, drugs, genes, or biologics. |
| Plaque Modification | Apply a voltage of about 100 V-2500 V, for about 5-500 |
| (Soft and Mixed Plaque) | pulses, using monophasic or biphasic waveforms (symmetric or |
| | asymmetric), with pulse durations of about 1 μs-10 ms, delivered |
| | in contact, non-contact, or hybrid modes, to alter plaque |
| | morphology, reduce stiffness, and improve vessel compliance, |
| | wherein, in vascular applications, a conductive fluid is delivered at |
| | a flow rate of about 1-30 mL/s (preferably 2-15 mL/s) to clear |
| | blood from the treatment site, improve electric field uniformity in |
| | heterogeneous plaque, and reduce thrombogenic conditions, and |
| | wherein the fluid may optionally include biologically active |
| | agents. |
| Perivascular or Tumor | Apply a voltage of about 200 V-3000 V, for about 10-1000 |
| Ablation | pulses, using monophasic or biphasic waveforms (symmetric or |
| | asymmetric), with pulse durations of about 1 μs-100 ms, |
| | delivered in contact, non-contact, or hybrid modes, to disrupt |
| | tumor cells and associated vasculature, wherein a fluid medium is |
| | optionally delivered to provide a conductive environment and/or to |
| | deliver therapeutic agents, chemotherapeutics, genes, or biologics |
| | to the treatment region. |
| Combined | Apply a voltage of about 100 V-3000 V, for about 5-1000 pulses, |
| Electroporation Therapy | using monophasic or biphasic waveforms (symmetric or |
| (Permeabilization + | asymmetric), with pulse durations of about 100 ns-100 ms, |
| Ablation) | delivered in contact, non-contact, or hybrid modes, wherein pulse |
| | parameters are modulated to achieve both reversible |
| | permeabilization and irreversible cytotoxic effects within different |
| | spatial regions or temporal phases of the target tissue, |
| | wherein a fluid medium is delivered before, during, or synchronized |
| | with pulse delivery to establish a controlled conductive environment |
| | and/or to enable localized delivery of therapeutic agents or |
| | biologics. |

### Reversible Electroporation

In FIG. 4A, the method 400a for reversible electroporation includes deploying (402a) an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, method 400a includes measuring the conductivity of the liquid medium (e.g., 104) in the blood vessel (e.g., 106), e.g., via the electrodes (e.g., 120). The method 400a may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method 400a includes establishing (402a) a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method 400a includes directing (406a) delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target reversible electroporation response.

Method 400a includes uniformly delivering (406a), for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce reversible electroporation and subsequent exfoliation of the target tissue as a tissue response to the pulsed electric field application, e.g., per Tables 2 or 3.

In some embodiments, method 400a may apply a voltage of about 50 V-1000 V, for about 1-200 pulses, using monophasic or biphasic waveforms (symmetric or asymmetric), with pulse durations of about 100 ns-10 ms, to induce controlled and reversible membrane permeabilization and facilitate intracellular delivery of therapeutic agents, drugs, or genetic material without substantial cell death, wherein a fluid medium may be delivered at a flow rate of about 1-30 mL/s (preferably 2-15 mL/s), the fluid comprising a conductive fluid, therapeutic agents, drugs, genes, proteins, cells, or combinations thereof, and configured to establish a localized conductive and/or delivery environment.

### Example Pulsed Field Ablation

In FIG. 4B, the method 400b for pulsed field ablation includes deploying (402b) an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, method 400b includes measuring the conductivity of the liquid medium (e.g., 104) in the blood vessel (e.g., 106), e.g., via the electrodes (e.g., 120). The method 400b may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method 400b includes establishing (402b) a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method 400b includes directing (406b) delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target pulsed field ablation response.

Method 400b includes uniformly delivering (406b), for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce pulsed field ablation and subsequent exfoliation of the target tissue as a tissue response to the pulsed electric field application, e.g., per Tables 2 or 3.

In some embodiments, method 400b may apply a voltage of about 50 V-1000 V, for about 1-200 pulses, using monophasic or biphasic waveforms (symmetric or asymmetric), with pulse durations of about 100 ns-10 ms, to induce controlled and reversible membrane permeabilization and facilitate intracellular delivery of therapeutic agents, drugs, or genetic material without substantial cell death, wherein a fluid medium may be delivered at a flow rate of about 1-30 mL/s (preferably 2-15 mL/s), the fluid comprising a conductive fluid, therapeutic agents, drugs, genes, proteins, cells, or combinations thereof, and configured to establish a localized conductive and/or delivery environment.

The exemplary pulsed electric field system, via introduction of the flow of conductivity-controlled medium, can address hemolysis by targeting the vascular lumen walls without causing damage to the blood and blood cells.

For pulsed field ablation, the method 400b may apply a voltage of about 100 V-3000 V, for about 5-1000 pulses, using monophasic or biphasic waveforms (symmetric or asymmetric), with pulse durations of about 100 ns-100 ms, delivered in contact, non-contact, or hybrid modes, to induce irreversible membrane disruption and targeted cell death with minimal or controlled thermal effects, wherein, in vascular applications, a conductive fluid is delivered at a flow rate of about 1-30 mL/s (preferably 2-15 mL/s) to partially or substantially displace blood, establish a localized conductive pathway, and reduce thrombus formation, and wherein the fluid may optionally comprise therapeutic or biologically active agents.

### Perivascular or Tumor Ablation

In FIG. 4C, the method 400c for perivascular or tumor ablation to induce a target vascular smooth muscle cell ablation response by deploying (402c) an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, method 400c includes measuring the conductivity of the liquid medium (e.g., 104) in the blood vessel (e.g., 106), e.g., via the electrodes (e.g., 120). The method 400c may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method 400c includes establishing (402c) a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method 400c includes directing (406c) delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target perivascular or tumor ablation response, e.g., by modulating endothelial cells but killing smooth muscle cells.

Method 400c includes uniformly delivering (406c), for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the perivascular or tumor ablation response as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

For perivascular or tumor ablation, the method 400c may apply a voltage of about 200 V-3000 V, for about 10-1000 pulses, using monophasic or biphasic waveforms (symmetric or asymmetric), with pulse durations of about 1 μs-100 ms, delivered in contact, non-contact, or hybrid modes, to disrupt tumor cells and associated vasculature, wherein a fluid medium is optionally delivered to provide a conductive environment and/or to deliver therapeutic agents, chemotherapeutics, genes, or biologics to the treatment region.

### Hyperplasia (NIH) Treatment/VSMC Modulation

In FIG. 4D, the method 400d for neointimal hyperplasia (NIH) treatment/VSMC modulation by deploying (402d) an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, method 400d includes measuring the conductivity of the liquid medium (e.g., 104) in the blood vessel (e.g., 106), e.g., via the electrodes (e.g., 120). The method 400d may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method 400d includes establishing (402d) a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method 400d includes directing (406d) delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target neointimal hyperplasia (NIH) treatment/VSMC modulation response.

Method 400d includes uniformly delivering (406d), for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the neointimal hyperplasia (NIH) treatment/VSMC modulation response as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

For neointimal hyperplasia (NIH) treatment/VSMC modulation, the method 400d may apply a voltage of about 100 V-2000 V, for about 10-500 pulses, using monophasic or biphasic waveforms (symmetric or asymmetric), with pulse durations of about 1 μs-10 ms, delivered in contact, non-contact, or hybrid modes, to selectively modulate or ablate vascular smooth muscle cells while preserving extracellular matrix and enabling endothelial recovery, where, in vascular applications, a conductive fluid is delivered at a flow rate of about 1-30 mL/s (preferably 2-15 mL/s) to partially or fully displace blood within the treatment region, reduce electrical conductivity heterogeneity, and minimize thrombus formation, and wherein the fluid may optionally comprise therapeutic agents, drugs, genes, or biologics.

### Plaque Modification.

In FIG. 4E, the method 400e for plaque modification includes deploying (402e) an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, method 400e includes measuring the conductivity of the liquid medium (e.g., 104) in the blood vessel (e.g., 106), e.g., via the electrodes (e.g., 120). The method 400e may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method 400e includes establishing (402e) a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method 400e includes directing (406e) delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target plaque modification response.

Method 400e includes uniformly delivering (406e), for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the plaque modification response as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

For plaque modification, the method 400e may apply a voltage of about 100 V-2500 V, for about 5-500 pulses, using monophasic or biphasic waveforms (symmetric or asymmetric), with pulse durations of about 1 μs-10 ms, delivered in contact, non-contact, or hybrid modes, to alter plaque morphology, reduce stiffness, and improve vessel compliance, wherein, in vascular applications, a conductive fluid is delivered at a flow rate of about 1-30 mL/s (preferably 2-15 mL/s) to clear blood from the treatment site, improve electric field uniformity in heterogeneous plaque, and reduce thrombogenic conditions, and wherein the fluid may optionally include biologically active agents.

Biological and therapeutic. For plaque-containing vessels, the fluid can improve field distribution in heterogeneous plaque. The conductive fluid may include one or more of: ions or electrolytes, pharmacological agents, chemotherapeutic agents, gene delivery vectors, proteins or cytokines, or cell-based therapies, optionally delivered in a spatially controlled manner using the envelope and fluid system. The system may provide controlled modulation of vascular smooth muscle cells within plaque regions while minimizing damage to surrounding structural components.

### Endothelial Cell Stimulation/Low-Intensity Electrical Stimulation

A method may be performed for endothelial cell stimulation/low-intensity electrical Stimulation that includes deploying an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, the method includes measuring the conductivity of the liquid medium (e.g., 104) in the blood vessel (e.g., 106), e.g., via the electrodes (e.g., 120). The method may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method includes establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method includes directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target endothelial cell stimulation/low-intensity electrical stimulation.

Method includes uniformly delivering, for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the endothelial cell stimulation/low-intensity electrical stimulation as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

The method may apply a voltage of about 100 V-2500 V, for about 5-500 pulses, using monophasic or biphasic waveforms (symmetric or asymmetric), with pulse durations of about 1 μs-10 ms, delivered in contact, non-contact, or hybrid modes, to alter plaque morphology, reduce stiffness, and improve vessel compliance, wherein, in vascular applications, a conductive fluid is delivered at a flow rate of about 1-30 mL/s (preferably 2-15 mL/s) to clear blood from the treatment site, improve electric field uniformity in heterogeneous plaque, and reduce thrombogenic conditions, and wherein the fluid may optionally include biologically active agents.

### Non-Thermal Ablation Via Electroporation (Includes Irreversible Electroporation (IRE) and Pulsed Field Ablation (PFA))

A method may be performed for non-thermal ablation via electroporation that includes deploying an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, the method includes measuring the conductivity of the liquid medium (e.g., 104) in the blood vessel (e.g., 106), e.g., via the electrodes (e.g., 120). The method may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method includes establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method includes directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target non-thermal ablation via electroporation response.

Method includes uniformly delivering, for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the non-thermal ablation via electroporation as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

The method may apply a voltage of about 100 V-3000 V, for about 5-1000 pulses, using monophasic or biphasic waveforms (symmetric or asymmetric), with pulse durations of about 100 ns-100 ms, delivered in contact, non-contact, or hybrid modes, to induce irreversible membrane disruption and targeted cell death with minimal or controlled thermal effects, wherein, in vascular applications, a conductive fluid is delivered at a flow rate of about 1-30 mL/s (preferably 2-15 mL/s) to partially or substantially displace blood, establish a localized conductive pathway, and reduce thrombus formation, and wherein the fluid may optionally comprise therapeutic or biologically active agents.

### Combined Electroporation Therapy (Permeabilization+Ablation)

A method may be performed for combined electroporation therapy that includes deploying an intravascular catheter (e.g., 102), e.g., as described in relation to FIGS. 1A-1D and 2A-2E, and 3A-3B, at least one fluid delivery lumen (e.g., 103) in a blood vessel, e.g., where the intravascular catheter (e.g., 102) comprising an expandable electrode assembly having a plurality of electrodes (e.g., 120) arranged in a basket or expanded configuration, wherein the plurality of electrodes comprises 4-12 electrodes. Each of the electrodes being (i) stowable within the catheter in a first configuration and (ii) expandable to a second configuration to conform to an inner wall of a blood vessel of varying diameter without occluding the blood vessel, and where the expandable electrode assembly is configured to be deployed within the blood vessel such that the electrodes are positioned in proximity to or in contact with the vessel wall.

The fluid delivery lumen (e.g., 103) is configured to be positionable into the blood vessel concurrent with the intravascular catheter without occluding the blood vessel to introduce a conductive fluid (e.g., an iso-osmolar conductive fluid, or biological fluid) into the blood vessel. In some embodiments, the method includes measuring the conductivity of the liquid medium (e.g., 104) in the blood vessel (e.g., 106), e.g., via the electrodes (e.g., 120). The method may include determining the conductivity ratio for the procedure, and introducing conductivity fluid until the measured conductivity is in the desired conductivity ratio, e.g., per Table 1. The operation may include removing liquid from the blood vessel, recirculating the liquid in the blood vessel while introducing fluid of greater or lower conductivity into the recirculation loop. In some embodiments, the recirculation loop may be heated or cooled to regulate the temperature of the liquid medium in the blood vessel.

The method includes establishing a localized conductive environment via a controller coupled to (i) the at least one fluid delivery lumen and the (ii) the plurality of electrodes to direct conductive fluid (e.g., via control of a pump or flow/pressure regulator) to the at least one fluid delivery lumen to partially or fully displace blood at a treatment site and establish the localized conductive environment. The operation may include removing liquid from the blood vessel and recirculating the liquid while introducing fluid of greater or lower conductivity into the recirculation loop. In some embodiments, the recirculation loop may be heated or cooled to regulate the temperature of the liquid medium in the blood vessel.

The method includes directing delivery of a plurality of electrical pulse waveforms within the conductive fluid environment via the controller to direct the plurality of electrical pulse waveforms to the tissue to penetrate a controlled depth into the vessel wall (e.g., directly to the tissue in a contact mode, through the conductive fluid in a non-contact mode, or in hybrid mode), wherein the plurality of electrical pulse waveforms applied through the conductive fluid environment and into the controlled depth of the vessel wall causes a target reversible electroporation response, and wherein the controller stores in memory a set of selectable waveforms (e.g., square, exponential, or user-defined shaped pulses) that are selectable by a user to direct delivery of the plurality of electrical pulse waveforms to induce the target combined electroporation therapy response.

Method includes uniformly delivering, for the conductivity of the liquid medium, an effective amount of electropulses to a target tissue (e.g., surface tissue) of the blood vessel to penetrate at least a pre-defined depth into the surface tissue to induce a tissue response of the target tissue of the blood vessel to induce the combined electroporation therapy response as a tissue response to the pulsed electric field application, e.g., per Tables 2 or 3.

The exemplary pulsed electric field system, via introduction of the flow of conductivity-controlled medium, can address hemolysis by targeting the vascular lumen walls without causing damage to the blood and blood cells.

The method may apply a voltage of about 100 V-3000 V, for about 5-1000 pulses, using monophasic or biphasic waveforms (symmetric or asymmetric), with pulse durations of about 100 ns-100 ms, delivered in contact, non-contact, or hybrid modes, wherein pulse parameters are modulated to achieve both reversible permeabilization and irreversible cytotoxic effects within different spatial regions or temporal phases of the target tissue, where a fluid medium is delivered before, during, or synchronized with pulse delivery to establish a controlled conductive environment and/or to enable localized delivery of therapeutic agents or biologics.

### Experimental Results and Additional Examples

A study was conducted to (i) develop an experimental system configured for intravascular pulsed electric field treatment as described herein and (ii) evaluate, in vitro, the effect of intravascular pulsed electric field (PEF) on endothelial cells (EC). The study also examined the effect on vascular permeability and elucidated signaling pathways mediating EC responses to PEF.

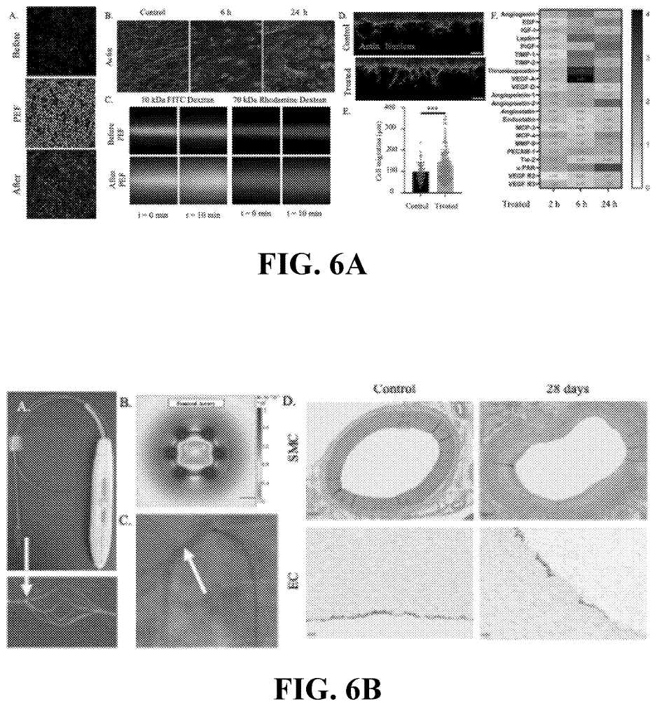

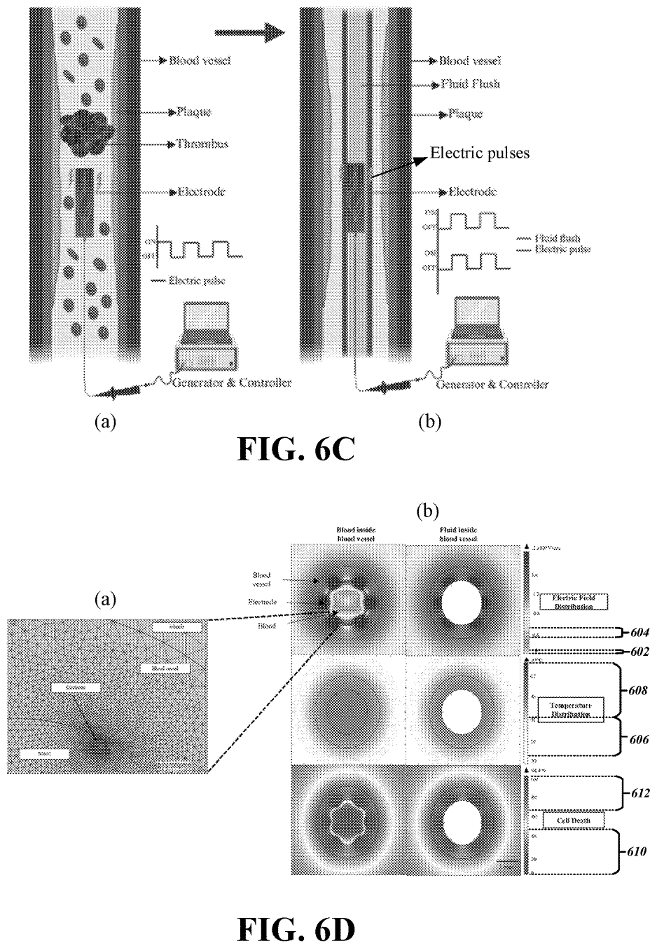

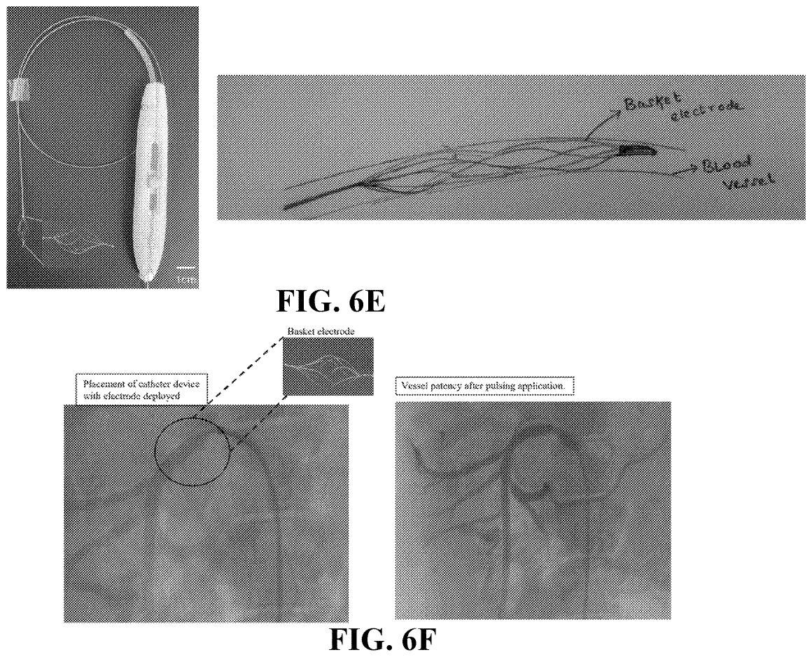

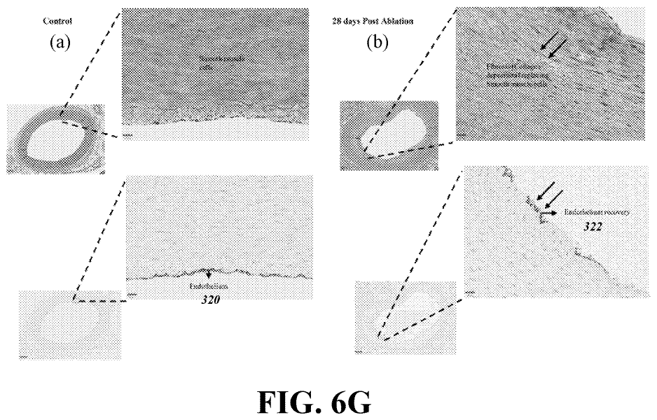

Intravascular pulsed electric field. FIG. 6A shows the effects of an intravascular pulsed electric field on endothelial cells. The study identified PEF waveforms with high specificity for EC that triggered rapid Ca²⁺ influx into the cytosol (see FIG. 6A, subpanel (a)). The Ca²⁺ influx activity led to actin cytoskeleton remodeling (see FIG. 6A, subpanel (b)) and to the internalization of gap and adherens junction proteins, resulting in a temporary loss of selective barrier function in EC monolayers (see FIG. 6A, subpanel (c)). The study also observed an elevation in the levels of pro-angiogenic factors and cytokines (see FIG. 6A, subpanel (f)), promoting EC motility (see FIG. 6A, subpanel (e)), and phenotypic normalization and vessel sprouting activity (see FIG. 6A, subpanel (d)). Cellular and molecular responses of EC to PEF stimulation could (i) enable localized, high-dose drug delivery with predictable kinetics and (ii) promote wound healing, re-endothelialization, and vessel formation. Therefore, the experimental system could be suitable for the therapy of chronic and malignant diseases associated with aberrant EC function.

System Configurations. FIG. 6B shows a non-occlusive basket catheter configured for endovascular PEF delivery to arteries within an object. The study used the catheter (see FIG. 6B, subpanel (a)) to deliver endovascular PEF to major arteries (see FIG. 6B, subpanel (c)) in 4 patient-sized Yorkshire swines (n=4), to evaluate the feasibility, safety, and effect of PEF on smooth-muscle cell (SMC).

In the study, angiographic imaging confirmed preservation of vessel patency, without acute adverse events. Histologic analysis of the vessel wall at ~48 hours post-treatment showed deep delivery of PEF into the vessel wall, confirming ablation of the SMC layer while preserving the basement membrane. Follow-up analysis of samples collected 28 days after treatment indicated complete endothelial recovery with durable elimination of underlying SMC (see FIG. 6B, subpanel (d)), establishing the safety and reproducibility of the experimental system's catheter-directed intravascular PEF delivery in major arteries.

The study further developed a basket electrode (e.g., wet electrode) coupled to the catheter for the experimental system to deliver PEF in luminal organs [8]. The basket electrode facilitated non-contact delivery of energy within tubular organs, where the study used differences in electrical conductivity gradients to focus effects on specific cell layers. The study also developed conductive fluid flushing for the experimental system, using a microcontroller-driven precision pump to infuse a conductive iso-osmolar fluid during energy delivery. Infusion of a fluid column could (i) keep the electrode wet for focusing PEF delivery to targeted EC or SMC layers in the vessel wall and (ii) reduce PEF exposure to blood, thereby minimizing hemolysis, microthrombi formation, and sequelae.

The study established finite element analysis (FEA) simulation models of intravascular electric field distribution (see FIG. 6B, subpanel (b)), based on angiograms of peripheral arterial disease (PAD) patient arteries and plaques, to determine the optimal PEF delivery configuration. The study used FEA simulations to (i) calculate the electrical properties of the fluid infused to generate the basket electrode and (ii) determine the optimal waveform sequencing to localize treatment effects to a targeted tissue layer. The study also used FEA simulations to (i) optimize the PEF waveform and the total energy deposited to mitigate thermal effects and (ii) predict the expected penetration and extent of treatment.

For efficient ablation, the experimental system could deliver, via the catheter and basket electrode, monophasic and biphasic pulses. A square wave could be used for monophasic PFA, with voltages of 100-3000V, pulse widths of 5-150 μs, and an application rate of 0.1-50 kHz. Depending on the desired result, the number of pulses could be set between 3 and 900. Symmetric or asymmetric square pulses could be used for biphasic PFA, with voltages of 100-3000V, pulse widths of 0.05 ns-20 μs, and interpulse or interburst delays of 5 ns-1s. The pulse number can range from 3 to 50,000, with bursts and frequencies adjustable to optimize exfoliation while minimizing thermal effects. The above-discussed parameters can be adjusted based on the specific arterial tissue and treatment requirements, ensuring that the selected pulse parameters induce the desired therapeutic effects.

Evaluation Setup. The study developed patient-sized organ-on-a-chip (OOC) vascular devices for investigating PEF in EC. The study updated the OOC devices to simulate PAD-like conditions by incorporating physiologically relevant flow and upsizing the devices to accept the experimental system's catheter for testing. The study included flow perturbations in the OOC devices to induce irregular vessel wall thickening and an aberrant EC layer. Inflammation in EC was stimulated by TNFα or LPS treatment, followed by infusion of activated monocytes to produce foam cells. The study developed simulation models (e.g., FEA simulation) of this OOC, where experiments were performed to confirm three outcomes with the experimental system: (i) stimulation of EC, recapitulating results on cytokine secretion and angiogenesis, (ii) ablation of SMC layer and studying impact on EC, and (iii) demonstration of bolus drug delivery to the vessel wall.

The study tested the experimental system in Yorkshire swine to validate the intraprocedural safety, the feasibility of PEF delivery, and the longitudinal tissue response. The popliteal and tibial arteries of the swine underwent PEF treatment to stimulate EC, ablation of SMC, and local delivery of a reporter dye, followed by bland balloon angioplasty without stenting. The contralateral side was held as a control undergoing angioplasty without PEF treatment. Swine were monitored clinically for treatment-related adverse events. Tissue was collected at 2 and 28 days post-treatment, where early samples were used to determine acute treatment effects and dye deposition patterns. The latter samples were used to compare tissue response, EC status, healing, and NIH between PEF-treated and control locations.

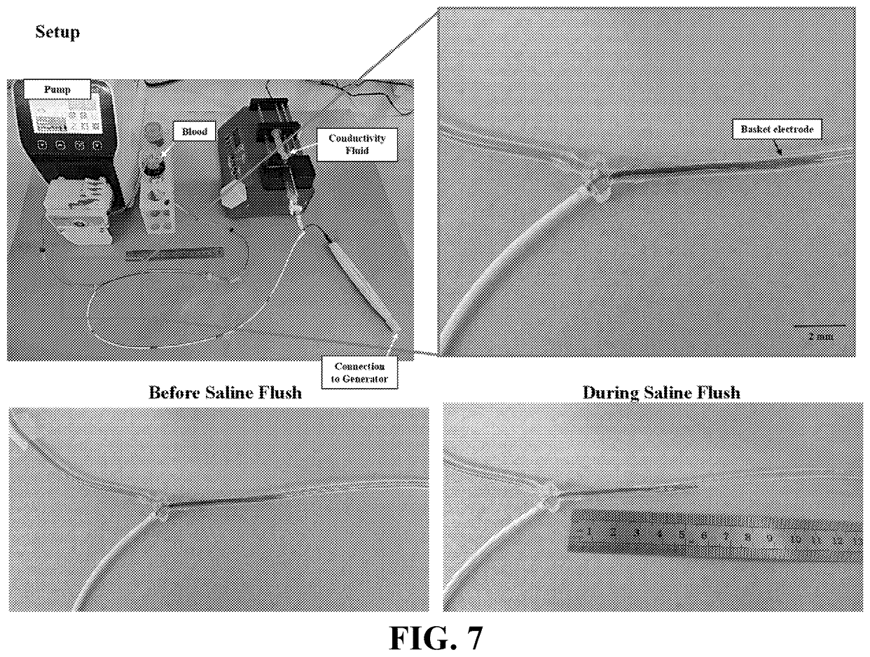

Pulsed Field Ablation Treatment. The study used the basket catheter to treat atherosclerotic lesions before stenting or to manage neointimal hyperplasia (NIH) after stent occlusion. By employing pulsed field ablation (PFA), the catheter delivered controlled electrical pulses to the vascular wall to reduce NIH and restenosis risk. The catheter had a self-expandable basket electrode (e.g., wet electrode) that could be deployed within an artery of variable diameter, enabling targeted vascular treatment at the site and providing an efficient, non-pharmacological alternative that eliminates the need for drug interventions. FIG. 7 shows the hardware configuration employed in the study.

Conductive fluid flushing was performed before pulse delivery to clear blood from the vessel, ensuring uniform electric-field distribution and reducing thrombus risk. FIG. 6C shows the effect of fluid flushing on thrombus prevention during pulsed field ablation with the catheter. In FIG. 6C, subpanel (a) shows that applying electric pulses in a blood-filled vessel leads to thrombus formation. In FIG. 6C, subpanel (b), flushing the vessel with fluid before pulse application prevents thrombus formation and ensures uniform treatment. The generator and controller unit delivered pulses to the basket electrode for controlled and targeted ablation.

FEA Simulations. The study used FEA simulations to refine the electrode configuration and predict the depth of tissue penetration during treatment. The study validated the simulations through in vitro and in vivo experiments. The simulations focused on electroporation effects in three arterial regions: the femoral artery, superior mesenteric artery, and renal artery, with and without blood. The wet electrode made contact at six evenly distributed points along the arterial wall. The electrode was 0.19 mm in diameter to ensure optimal vascular contact and uniform field distribution. To analyze the effects on vascular tissue, the simulations were conducted under 2 pulsed-field ablation (PFA) conditions: (i) blood inside the blood vessel, and (ii) fluid (e.g., saline) inside the blood vessel to reduce thrombus risk and improve electric field uniformity.

The electrical behavior of the experimental system was governed by Maxwell's Equations, with a focus on electrostatic fields, which simplified Laplace's Equation in a steady-state condition. The study maintained a constant current density throughout the system. Boundary conditions included applying DC voltage on the electrodes, while a distal ground pad at the perimeter of the simulation domain served as the reference ground.

FIG. 6D shows FEA simulations to analyze the effects of pulsed-field ablation in a blood vessel. In FIG. 6D, subpanel (a) shows a meshed simulation model of the electrode, blood vessel, and surrounding tissue. In FIG. 6D, subpanel (b), the top row shows the electric field distribution, where shade 602 indicates lower field intensity and shade 604 indicates higher intensity around the electrode. The middle row shows the temperature distribution, where areas with shades 606 and 608 represent regions of increased heating. The bottom row depicts the extent of cell death, where areas with shade 610 indicate high cell death due to ablation and areas with shade 612 show minimal effect. The comparison between the two columns highlights that fluid flushing ensures uniform treatment inside the blood vessel, preventing disturbances and improving ablation efficiency.

The simulation results in FIG. 6D show that the electric field strength (EFS) varied across arterial locations, whereas the electrode provided a uniform field. In the presence of blood, the field penetration decreased due to higher conductivity, whereas in saline-filled vessels, the electric field was evenly distributed. The simulations also examined the temperature distribution, revealing localized heating near the electrodes due to Joule heating during ablation without fluid infusion. While electroporation was considered non-thermal, temperature variations depended on artery type and conductivity, with saline-filled vessels exhibiting reduced heating compared to blood-filled conditions.

The extent of cell death correlated with the electric field intensity, confirming effective ablation in the NIH-prone arterial regions. Higher conductivity in blood-filled vessels resulted in a broader but weaker electric field, whereas saline-filled vessels exhibited a homogeneous, penetrating electric field distribution into the vessel wall, thereby enhancing electroporation efficiency.

Overall, the FEA simulations confirmed that the electrode delivered pulsed electric fields to the targeted vascular regions. The presence of blood affected the electric field distribution and cell death patterns, highlighting the benefits of saline flushing to optimize treatment uniformity. The simulation results helped optimize electrode design and pulse parameters for intravascular electroporation, ensuring safe and effective prevention of NIH.

In Vivo Assessment of the Catheter. FIG. 6E shows a non-occlusive basket catheter having a self-expandable basket electrode. The catheter could be inserted into a blood vessel, allowing precise positioning of the self-expandable basket electrode at its distal end. Upon fluid flush, the electrode delivered electric pulses for targeted ablation. The catheter was also configured to deploy or retract the electrode.

The study assessed the feasibility and safety of the catheter and associated basket electrode for transarterial pulsed field ablation (PFA) in Yorkshire swine. The study targeted three arteries: the superior mesenteric artery, renal artery, and common femoral artery. To minimize thrombus formation, saline flushing was performed before electrical pulse application to clear the treatment site of blood. Example electric pulses employed in the study had a pulse voltage of 2000V, a pulse width of 90 μs, and 70 pulses at 1.5 Hz, which represented standard values for non-thermal irreversible electroporation (IRE). The study conducted digital subtraction angiography (DSA) post-treatment to assess vessel integrity and identify potential complications such as dissection, thrombosis, or perforation. The study euthanized the swines for 4 hours, 2 days, and 28 days post-treatment for histopathological evaluation of the treated arteries.

FIG. 6F shows angiographic imaging (e.g., fluoroscopic imaging) of the pulsed-field ablation. In FIG. 6F, subpanel (a), the catheter was positioned within the blood vessel, and after fluid infusion, the basket electrode was deployed at the target site. In FIG. 6F, subpanel (b), the vascular anatomy was visualized during the application of electric pulses. The imaging sequence in FIG. 6F showed the process of electrode placement and activation for precise PFA delivery. The study performed pulsed-field ablation in 19 vessels across 5 swine, with no immediate vascular damage detected on imaging (see FIG. 6F).

FIG. 6G shows a histological analysis of smooth muscle remodeling and endothelial regeneration after pulsed-field ablation. In FIG. 6G, subpanel (a) (shown as “control”), smooth muscle cells (SMCs) and an intact endothelial layer were visible. In FIG. 6G, subpanel (b) (shown as “28 days post ablation”), fibrosis replaced necrotic SMCs, indicating structural remodeling. In FIG. 6G, subpanel (a) further shows a continuous endothelial lining 620, while subpanel (b) further shows endothelial regeneration 622 after 28 days. High-magnification images confirm selective SMC ablation with preserved endothelial recovery, preventing neointimal hyperplasia.

Histopathological analysis in FIG. 6G confirmed precise and controlled electroporation-induced cell death within the intimal and medial layers of the arterial wall, while the basement membrane and adventitia remained intact, preserving vascular structure. The extent of ablation varied by anatomical site, with skeletal muscle penetration reaching 5 mm. The histopathological analysis also showed endothelial recovery following PFA treatment, supporting the long-term efficacy of PFA in mitigating NIH.

The evaluation results demonstrated that transarterial pulsed-field ablation using the non-occlusive catheter with the basket electrode was both feasible and safe, effectively ablating targeted arterial tissue without compromising vascular integrity. The non-occlusive catheter presents a promising therapeutic approach for managing neointimal hyperplasia (NIH), restenosis, and peri-vascular tumor infiltration.

### Discussion

Peripheral arterial disease (PAD) is characterized by atherosclerotic occlusion of the lower limb arteries, leading to ischemia, pain, unhealing ulcers, and limb loss [1]. Oral medication with anticoagulant drugs or statins provides limited control. The standard of care is endovascular therapy with angioplasty using drug-eluting balloons or stents [2]. Drugs (e.g., Paclitaxel, Everolimus) are necessary after stent placement to inhibit neointimal hyperplasia (NIH), the proliferation of smooth muscle cells (SMCs), which can obscure vessel patency [3]. Despite advances in stent manufacturing and drug-eluting stent formulations, restenosis occurred in 30-80% of patients within 2 years [2].

EC dysfunction is a PAD that contributes to pathobiology, disease progression, and symptom orchestration [4]. EC in PAD exhibit an inflammatory phenotype with reduced capacity for mechanotransduction, impacting normal barrier function. Alterations to endothelial nitric oxide synthase (eNOS) and vascular endothelial growth factor (VEGF) signaling in EC underlie ischemia, thrombus formation, and vessel occlusion. After angioplasty and stenting, slow endothelialization contributes to NIH and ongoing inflammation [5]. Paclitaxel, used to control SMC proliferation, is toxic to EC, exacerbating this feedback loop [6]. The exemplary system is configured to address unmet clinical needs in PAD therapy by modulating EC function via PEF stimulation.

PAD is a chronic health condition afflicting 250 million patients worldwide, increasing by 10 million each year [7]. The market for endovascular devices in PAD treatment is estimated at $3-6 billion annually, with a 8% compound annual growth rate. The addressable market for the exemplary system would mirror that of drug eluting balloon and stents, estimated at $873 million in 2024 and expected to grow to $1.74 billion by 2032. Due to demographic factors, 45% of the PAD patient population can be in the USA. The total healthcare footprint of PAD is sizeable due to morbidity, loss of mobility, the need for repeated re-intervention, and poor long-term outcomes. Thus, successful treatment of PAD can unlock healthcare savings beyond the immediate device market. Improved drug delivery to the vessel wall, preparation of the vessel prior to stenting, non-mechanical plaque removal, and devices that promote wound healing are consensus clinical needs that form the basis of development activities. There are emerging devices attempting to address the above-discussed individual needs, e.g., resorbable drug-eluting stents (e.g., R3 Vascular), covered stents to improve outcomes (e.g., BD Inc.), and lithotripsy for plaque removal (e.g., Fastwave Medical). However, current market solutions fall short of the integrated treatment approach achievable with the exemplary system.

The exemplary system can have various applications. By utilizing pulsed-field ablation (PFA), the exemplary system can provide margins for tumors adjacent to or infiltrating blood vessels, thereby allowing safe surgical resection. Additionally, PFA can alter the endothelial cell barrier, enabling the targeted delivery of drugs, genes, or other materials directly into blood vessels, thus enhancing the effectiveness of therapies for vascular diseases and tumors. Further, the exemplary system can promote controlled vascular remodeling in chronic conditions by selectively modifying blood vessel architecture, potentially promoting healthier blood flow and tissue healing.

### CONCLUSION

The construction and arrangement of the systems and methods, as shown in the various implementations, are illustrative only. Although only a few implementations have been described in detail in this disclosure, many modifications are possible (e.g., variations in sizes, dimensions, structures, shapes, proportions of the various elements, parameter values, mounting arrangements, materials, colors, orientations, etc.). For example, the positions of elements may be reversed or otherwise rearranged, and the nature or number of discrete elements or positions may be altered. Accordingly, all such modifications are intended to be included within the scope of the present disclosure. The order or sequence of any process or method steps may be varied or resequenced in alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special-purpose computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a general-purpose or special-purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium; thus, any such connection is properly termed a machine-readable medium. Combinations of the above are also included within the scope of machine-readable media. Machine-executable instructions include, for example, instructions and data that cause a general-purpose computer, special-purpose computer, or special-purpose processing machine to perform a certain function or group of functions.

It is to be understood that the methods and systems are not limited to specific synthetic methods, specific components, or particular compositions. It is also to be understood that the terminology used herein is for the purpose of describing particular implementations only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint and independently of the other endpoint.

“Optional” or “optionally” means that the subsequently described event or circumstance may or may not occur and that the description includes instances where said event or circumstance occurs and instances where it does not.

Throughout the description and claims of this specification, the word “comprise” and variations of the word, such as “comprising” and “comprises,” mean “including but not limited to,” and are not intended to exclude, for example, other additives, components, integers, or steps. “Exemplary” means “an example of” and is not intended to convey an indication of a preferred or ideal implementation. “Such as” is not used in a restrictive sense but for explanatory purposes.

Disclosed are components that can be used to perform the disclosed methods and systems. These and other components are disclosed herein, and it is understood that when combinations, subsets, interactions, groups, etc. of these components are disclosed while specific reference to each various individual and collective combinations and permutations of these may not be explicitly disclosed, each is specifically contemplated and described herein, for all methods and systems. This applies to all aspects of this application, including, but not limited to, steps in disclosed methods. Thus, if there are a variety of additional steps that can be performed, it is understood that each of these additional steps can be performed with any specific implementation or combination of implementations of the disclosed methods.

The following patents, applications, and publications, as listed below and throughout this document, are hereby incorporated by reference in their entirety herein.
[1] Barton, M. Mechanisms and therapy of atherosclerosis and its clinical complications. Curr Opin Pharmacol 13, 149-153 (2013).
[2] Collins, M. J. et al. Therapeutic strategies to combat neointimal hyperplasia in vascular grafts. Expert Rev Cardiovasc Ther 10, 635-47 (2012).
[3] Ebert, M. L. A. et al. Animal Models of Neointimal Hyperplasia and Restenosis. JACC Basic Transl Sci 6, 900-917 (2021).
[4] Jukema, J. W., Verschuren, J. J. W., Ahmed, T. A. N., & Quax, P. H. A. Restenosis after PCI. Part 1: pathophysiology and risk factors. Nat Rev Cardiol 9, 53-62 (2012).
[5] Looser, P. M., Kim, L. K. & Feldman, D. N. In-Stent Restenosis: Pathophysiology and Treatment. Curr Treat Options Cardiovasc Med 18, 10 (2016).
[6] Changal, K. H. et al. Drug-Eluting Stents Versus Bare-Metal Stents in Large Coronary Artery Revascularization: Systematic Review and Meta-Analysis. Cardiovascular Revascularization Medicine 23, 42-49 (2021).
[7] Donisan, T., Madanat, L., Balanescu, D. V., Mertens, A. & Dixon, S. Drug-Eluting Stent Restenosis: Modern Approach to a Classic Challenge. Curr Cardiol Rev 19, (2023).
[8] Habib, A. & Finn, A. V. Endothelialization of drug-eluting stents and its impact on dual antiplatelet therapy duration. Pharmacol Res 93, 22-27 (2015).
[9] Owens, C. D. Adaptive changes in autogenous vein grafts for arterial reconstruction: clinical implications. J Vasc Surg 51, 736-46 (2010).
[10] Li, L., Terry, C. M., Shiu, Y.-T. E. & Cheung, A. K. Neointimal hyperplasia associated with synthetic hemodialysis grafts. Kidney Int 74, 1247-1261 (2008).
[11] Lawson, J. H., Niklason, L. E. & Roy-Chaudhury, P. Challenges and novel therapies for vascular access in haemodialysis. Nat Rev Nephrol 16, 586-602 (2020).
[12] Newby, A. C. & Zaltsman, A. B. Molecular mechanisms in intimal hyperplasia. J Pathol 190, 300-309 (2000).
[13] Myit, S. et al. Different Growth Properties of Neointimal and Medial Smooth Muscle Cells in Response to Growth Factors. J Vasc Res 40, 97-104 (2003).
[14] Pal, N., Din, J. & O'Kane, P. Contemporary Management of Stent Failure: Part One. Interventional Cardiology Review 14, 10-16 (2019).
[15] Omeh D J, S. E. Restenosis of Stented Coronary Arteries. (Treasure Island (FL): StatPearls Publishing (2023).
[16] Cao, Z. et al. The factors influencing the efficiency of drug-coated balloons. Front Cardiovasc Med 9 (2022).
[17] Eikelboom, J. W., Hirsh, J., Spencer, F. A., Baglin, T. P., & Weitz, J. I. Antiplatelet Drugs. Chest 141, e89S-e119S (2012).
[18] London: National Institute for Health and Care Excellence. Evidence Review for Statins: Efficacy and Adverse Effects: Cardiovascular Disease: Risk Assessment and Reduction, Including Lipid Modification: Evidence Review. (2023).
[19] Von der Leyen, H. E. et al. Gene therapy inhibiting neointimal vascular lesion: in vivo transfer of endothelial cell nitric oxide synthase gene. Proceedings of the National Academy of Sciences 92, 1137-1141 (1995).
[20] Kong, D. et al. Enhanced Inhibition of Neointimal Hyperplasia by Genetically Engineered Endothelial Progenitor Cells. Circulation 109, 1769-1775 (2004).
[21] Pala, R., Anju, V., Dyavaiah, M., Busi, S., & Nauli, S. M. Nanoparticle-Mediated Drug Delivery for the Treatment of Cardiovascular Diseases. Int J Nanomedicine Volume 15, 3741-3769 (2020).
[22] Strobel, H. A., Qendro, E. I., Alsberg, E., & Rolle, M. W. Targeted Delivery of Bioactive Molecules for Vascular Intervention and Tissue Engineering. Front Pharmacol 9, (2018).
[23] Alam, M., Hsu, T., Dover, J. S., Wrone, D. A., & Arndt, K. A. Nonablative laser and light treatments: Histology and tissue effects—A Review. Lasers Surg Med 33, 30-39 (2003).
[24] Ni, Y., Mulier, S., Miao, Y., Michel, L. & Marchal, G. A review of the general aspects of radiofrequency ablation. Abdom Imaging 30, 381-400 (2005).
[25] Brace, C. L. Microwave Tissue Ablation: Biophysics, Technology, and Applications. Crit Rev Biomed Eng 38, 65-78 (2010).
[26] Erinjeri, J. P. & Clark, T. W. I. Cryoablation: Mechanism of Action and Devices. Journal of Vascular and Interventional Radiology 21, S187-S191 (2010).
[27] Lang, B. H.-H. & Wu, A. L. H. High intensity focused ultrasound (HIFU) ablation of benign thyroid nodules—a systematic review. J Ther Ultrasound 5, 11 (2017).
[28] Kübler, A. C. Photodynamic therapy. Medical Laser Application 20, 37-45 (2005).
[29] Popa, M. A. et al. Myocardial injury and inflammation following pulsed-field ablation and very high-power short-duration ablation for atrial fibrillation. J Cardiovasc Electrophysiol 35, 317-327 (2024).
[30] Shah, D. R., Green, S., Elliot, A., McGahan, J. P., & Khatri, V. P. Current oncologic applications of radiofrequency ablation therapies. World J Gastrointest Oncol 5, 71-80 (2013).
[31] Lubner, M. G., Brace, C. L., Hinshaw, J. L., & Lee, F. T. Microwave Tumor Ablation: Mechanism of Action, Clinical Results, and Devices. Journal of Vascular and Interventional Radiology 21, S192-S203 (2010).
[32] FINGAR, V. H. Vascular Effects of Photodynamic Therapy. J Clin Laser Med Surg 14, 323-328 (1996).
[33] Dinc, R. A review of the current state in neointimal hyperplasia development following endovascular intervention and minor emphasis on new horizons in immunotherapy. Transl Clin Pharmacol 31, 191 (2023).
[34] Lanza, G. M. et al. Targeted Antiproliferative Drug Delivery to Vascular Smooth Muscle Cells With a Magnetic Resonance Imaging Nanoparticle Contrast Agent. Circulation 106, 2842-2847 (2002).
[35] Franzone, A. et al. The role of atherectomy in the treatment of lower extremity peripheral artery disease. BMC Surg 12, S13 (2012).
[36] Goel, S. A., Guo, L.-W., Liu, B. & Kent, K. C. Mechanisms of post-intervention arterial remodelling. Cardiovasc Res 96, 363-371 (2012).
[37] Wang, L.-H., Jiang, Y., Sun, C.-H., Chen, P.-T. & Ding, Y.-N. Advancements in the application of ablative therapy and its combination with immunotherapy in anti-cancer therapy. Biochimica et Biophysica Acta (BBA)—Reviews on Cancer 1880, 189285 (2025).
[38] Michael B. Sano, Rafael V. Davalos, John L. Robertson, Paulo A. Garcia, Robert E. Neal. Irreversible electroporation using tissue vasculature to treat aberrant cell masses or create tissue scaffolds. (2020).
[39] Jourabchi, N., Beroukhim, K., Tafti, B. A., Kee, S. T., & Lee, E. W. Irreversible electroporation (NanoKnife) in cancer treatment. Gastrointestinal Intervention 3, 8-18 (2014).
[40] Iyengar, S. K., Iyengar, S. & Srivathsan, K. The promise of pulsed field ablation and the challenges ahead. Front Cardiovasc Med 10, (2023).
[41] Evans, C. E., Iruela-Arispe, M. L. & Zhao, Y.-Y. Mechanisms of Endothelial Regeneration and Vascular Repair and Their Application to Regenerative Medicine. Am J Pathol 191, 52-65 (2021).
[42] Meng, F., Jin, S., & Liu, N. Cardiac selectivity in pulsed field ablation. Curr Opin Cardiol 40, 37-41 (2025).
[43] Maor, E., Ivorra, A., Mitchell, J. J., & Rubinsky, B. Vascular Smooth Muscle Cells Ablation with Endovascular Nonthermal Irreversible Electroporation. Journal of Vascular and Interventional Radiology 21, 1708-1715 (2010).
[44] Chinyere, I. R., Mori, S., & Hutchinson, M. D. Cardiac blood vessels and irreversible electroporation: findings from pulsed field ablation. Vessel Plus (2024) doi: 10.20517/2574-1209.2023.80.

1. A system for intravascular pulsed electric field treatment, comprising:
an intravascular catheter comprising an expandable electrode assembly having a plurality of electrodes arranged in a basket configuration, wherein the plurality of electrodes comprises 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and
at least one fluid delivery lumen, including a fluid delivery lumen, configured to be positionable into the blood vessel concurrent with the intravascular catheter without occluding the blood vessel to introduce a conductive fluid into the blood vessel; and
a controller coupled to (i) the at least one fluid delivery lumen and the (ii) the plurality of electrodes,
wherein the controller is configured to direct conductive fluid to the at least one fluid delivery lumen to partially or fully displace blood at a treatment site and establish a localized conductive environment
wherein the controller is configured to direct delivery of a plurality of electrical pulse waveforms within the conductive fluid environment to direct the plurality of electrical pulse waveforms to penetrate a controlled depth into the vessel wall, wherein the plurality of electrical pulse waveforms applied through the conductive fluid environment and into the controlled depth of the vessel wall causes a target tissue response, and wherein the controller stores in memory a set of selectable waveforms that are selectable by a user to direct delivery of the plurality of electrical pulse waveforms to induce the target tissue response.

2. The system of claim 1, wherein the fluid delivery lumen is integrated within the intravascular catheter as a multi-lumen catheter.

3. The system of claim 1 further comprising:
a second catheter configured to be positionable into the blood vessel concurrent with the intravascular catheter without occluding the blood vessel to introduce a second conductive fluid into the blood vessel,
wherein the controller is configured to direct conductive fluid to the second catheter, wherein delivery of the conductive fluid by the fluid delivery lumen and the second catheter partially or fully displace blood at the treatment site and establish the localized conductive environment.

4. The system of claim 1, wherein the intravascular catheter is configured with a catheter arrangement selected from the group consisting of a coaxial configuration, a concentric configuration, a parallel configuration, and an independently navigable configuration.

5. The system of claim 1, wherein the plurality of electrodes is configured as an electrode assembly of symmetrically arranged electrodes.

6. The system of claim 1, wherein the controller is configured and selectable to direct delivery of the plurality of electrical pulse waveforms to all of the plurality of electrodes simultaneously.

7. The system of claim 1, wherein the controller is configured and selectable to direct delivery of the plurality of electrical pulse waveforms to each of the plurality of electrodes in a pre-defined sequence.

8. The system of claim 1, wherein the plurality of electrodes of the intravascular catheter are self-expanding upon being deployed from the intravascular catheter.

9. The system of claim 1, wherein the intravascular catheter is coupled to a handle, the handle having a manual actuator coupled to a mechanical connection to the plurality of electrodes, the manual actuator being adjustable to mechanically adjust a desired treatment length and diameter within a given vessel.

10. The system of claim 1, wherein the intravascular catheter is configured to stow the plurality of electrodes, including a central shaft electrode, in a stowed central shaft electrode configuration and to deploy the plurality of electrodes, including the central shaft electrode, in a deployed central shaft electrode configuration.

11. The system of claim 1, wherein the intravascular catheter is coupled to an expandable basket, wherein the intravascular catheter is configured to stow the plurality of electrodes in a stowed central shaft electrode configuration and to deploy the plurality of electrodes in a deployed central shaft electrode configuration, and wherein the plurality of electrodes are extendable within an interior region defined by the expandable basket in an expandable basket electrode configuration.

12. The system of claim 1, wherein the controller is coupled to a plurality of monopolar electrodes as the plurality of electrodes in a monopolar configuration.

13. The system of claim 1, wherein the controller is coupled to a plurality of radial field electrodes as the plurality of electrodes in a radial field configuration.

14. The system of claim 1, wherein the controller is coupled to a plurality of radial field electrodes as the plurality of electrodes in a radial field configuration, wherein surrounding basket electrodes of the plurality of electrodes act with opposite polarity to generate a radially distributed electric field.

15. The system of claim 1, wherein the controller is coupled to a plurality of bipolar configuration electrodes as the plurality of electrodes in a bipolar configuration, wherein adjacent or alternating basket electrodes of the plurality of electrodes.

16. The system of claim 1, wherein the controller is coupled to a plurality of hybrid configuration electrodes in a hybrid configuration.

17. The system of claim 1 comprising an imaging system configured to perform intravascular imaging.

18. The system of claim 1 comprising:
a sensor, wherein the controller is configured to adjust pulse, electrode position, and/or fluid parameters in real time based on sensor feedback.

19. A method comprising:
deploying an intravascular catheter at least one fluid delivery lumen in a blood vessel,
wherein the intravascular catheter comprising an expandable electrode assembly having a plurality of electrodes arranged in a basket configuration, wherein the plurality of electrodes comprises 4-12 electrodes, each of the electrodes being (i) stowable within the catheter in a first configuration and (ii) expandable to a second configuration to conform to an inner wall of a blood vessel of varying diameter without occluding the blood vessel, and wherein the expandable electrode assembly is configured to be deployed within the blood vessel such that the electrodes are positioned in proximity to or in contact with the vessel wall; and
wherein the at least one fluid delivery lumen includes a fluid delivery lumen and is configured to be positionable into the blood vessel concurrent with the intravascular catheter without occluding the blood vessel to introduce a conductive fluid into the blood vessel; and
establishing a localized conductive environment via a controller coupled to (i) the at least one fluid delivery lumen and the (ii) the plurality of electrodes to direct conductive fluid to the at least one fluid delivery lumen to partially or fully displace blood at a treatment site and establish the localized conductive environment,
directing delivery of a plurality of electrical pulse waveforms within the conductive fluid environment via the controller to direct the plurality of electrical pulse waveforms to penetrate a controlled depth into the vessel wall, wherein the plurality of electrical pulse waveforms applied through the conductive fluid environment and into the controlled depth of the vessel wall causes a target tissue response, and wherein the controller stores in memory a set of selectable waveforms that are selectable by a user to direct delivery of the plurality of electrical pulse waveforms to induce the target tissue response.

20. The method of claim 19, wherein the plurality of electrodes are deployed in a non-contact or a hybrid mode for electroporation, pulsed field ablation, vascular smooth muscle cell ablation, or reduction of neointimal hyperplasia as the target tissue response.

21.-27. (canceled)
